(19) United States
(12) Patent Application Publication
(10) Pub. No.: US 2019/0374474 A1
(43) Pub. Date: Dec. 12, 2019

(54) PHARMACEUTICAL COMPOSITIONS AND METHODS FOR WEIGHT LOSS

(71) Applicant: WELLESLEY PHARMACEUTICALS, LLC, Newtown, PA (US)

(72) Inventor: David A. DILL, Newton, PA (US)

(21) Appl. No.: 16/005,096

(22) Filed: Jun. 11, 2018

(51) Int. Cl. A61K 9/28; A61K 31/167; A61K 45/06; A61K 31/192; A61P 3/04

(52) U.S. Cl. CPC A61K 9/2893; A61K 31/167; A61P 3/04; A61K 31/192; A61K 9/2833; A61K 45/06

(57) ABSTRACT

Compositions and methods for reducing body weight in a subject are disclosed. In one aspect, a method for reducing body weight in a subject comprises: administering to a subject in need thereof a pharmaceutical composition comprising acetaminophen in an amount sufficient for reducing body weight, wherein the acetaminophen is administered orally at a daily dose of 5 mg to 2000 mg, and wherein the composition is formulated in a controlled release formulation. The acetaminophen may be administered alone or in combination with an anti-obesity drug, an appetite suppressant, a diacylglycerol acyltransferase (DGAT) inhibitor, a lipase inhibitor, a onsteroidal anti-inflammatory drugs (NSAIDs), a Type II diabetes agents, anti-insomnia agents or a combination thereof.

### FIELD

The present invention relates to compositions and methods for reducing body weight in a subject. More particularly, it relates to a method for reducing body weight comprising administering of an effective amount of a controlled release formulation comprising acetaminophen, alone or in combination with one or more weight reducing agents.

### BACKGROUND

Obesity is a growing epidemic in the United States that is in part caused by diet and lack of exercise. Approximately, 97 million Americans, or about 61 percent, are overweight or obese. Diets having a high fat content as well as the repeated ingestion of refined foods and sugars coupled with low fiber and vegetable intake contribute to the obesity epidemic. Diet, lack of exercise and the natural aging process causes deterioration in the manner in which the body metabolizes blood glucose. When the body cannot properly metabolize blood glucose, there is a tendency to store glucose as fat. There is a connection between insulin resistance and increased visceral adiposity, thus when glucose regulation is unbalanced, a greater propensity for adiposity exists. There is also a link between obesity and several diseases such as, heart disease, hypertension, Type II diabetes, and insulin resistance.

Many current weight loss strategies require significant limitations on the amount of caloric intake and/or increase in metabolizing the current caloric intake or stored fat which is generally done through aerobic exercise to achieve weight loss. While these are effective strategies for weight loss, many people find it difficult to start an exercise regimen and continue with reduced caloric intake. Furthermore, many adults consume coffee, typically at least one cup of coffee a day.

Accordingly, there is a need for simple and inexpensive means for reducing body weight in an individual without significant side effects. The inventor of the present application has unexpectedly discovered that administration of a pharmaceutical composition comprising acetaminophen can reduce body weight in a subject.

### SUMMARY

In one aspect, a method for reducing body weight in a subject, comprises: administering to a subject in need thereof a pharmaceutical composition comprising acetaminophen in an amount sufficient for reducing body weight, wherein the acetaminophen is administered orally at a daily dose of 5 mg to 2000 mg, and wherein the composition is formulated in a controlled release formulation.

In one embodiment, the acetaminophen is administered orally at a daily dose of 50 mg to 500 mg. In another embodiment, the acetaminophen is administered orally at a daily dose of 250 mg to 500 mg.

In one embodiment, the acetaminophen is formulated for extended-release, immediate-release, delayed release or a combination thereof.

In one embodiment, the acetaminophen is formulated for extended-release, whereby the acetaminophen is embedded in a matrix of insoluble substance(s).

In another embodiment, the acetaminophen is formulated in an extended =-release formulation, wherein the extended-release formulation comprises a water soluble or water-swellable matrix-forming polymer, a polymer controlling release of acetaminophen by dissolution controlled release and/or is coated with an enteric coating.

In another embodiment, the composition further comprises one or more additional active agents selected from the group consisting of anti-obesity drugs, appetite suppressants, diacylglycerol acyltransferase (DGAT) inhibitors, lipase inhibitors, nonsteroidal anti-inflammatory drugs (NSAIDs), Type II diabetes agents, anti-insomnia agents and combinations thereof.

In another embodiment, the composition is formulated to release the one or more additional active agents by immediate release, extended release, delayed release, or a combination thereof.

In a particular embodiment, the one or more additional active agents comprise at least one anti-obesity drug.

In another embodiment, the one or more additional active agents comprise at least one appetite suppressant.

In another embodiment, the one or more additional active agents comprise at least one DGAT inhibitor.

In another embodiment, the one or more additional active agents comprise at least one lipase inhibitor.

In another embodiment, the one or more additional active agents comprise at least one NSAID.

In another embodiment, the one or more additional active agents comprise at least one Type II diabetes agent.

In a particular embodiment, the composition comprises acetaminophen in combination with ibuprofen.

In another embodiment, each of the acetaminophen and ibuprofen is administered in an extended-release formulation.

In a further embodiment, the acetaminophen and ibuprofen are the only active therapeutic agents in the composition.

In another aspect, a method of manufacturing a pharmaceutical composition for reducing body weight comprises the steps of forming a first mixture having a first active agent formulated for immediate release and a second active agent formulated for extended release; coating the first mixture with a delayed release coating to form a core structure; and then coating the core structure with a second mixture comprising a third active agent formulated for immediate release and a fourth active agent formulated for extended release.

In another embodiment, the method of manufacturing the pharmaceutical composition comprises the steps of forming a core structure comprising a first active agent formulated for immediate release and a second active agent formulated for extended release, coating the core structure with a delayed release coating to form a coated core structure, and mixing the coated core structure with a third active agent formulated for immediate release and a fourth active agent formulated for extended release, wherein the first, second, third and fourth active agents can be any of the active agents described above.

In another embodiment, the method of manufacturing the pharmaceutical composition comprises the steps of forming a core structure comprising a first active agent formulated for immediate release and a second active agent formulated for extended release, coating the core structure with a delayed release coating to form a coated core structure, mixing the coated core structure with a third agent formulated for immediate release and a fourth agent formulated for extended release.

In another embodiment, the method of manufacturing the pharmaceutical composition comprises the steps of forming a core structure comprising a first active agent formulated for immediate release and a second active agent formulated for extended release; coating the core structure with a delayed release coating to form a coated core structure; mixing the coated core structure with a third active agent formulated for immediate release and a fourth active agent formulated for extended release to form a final mixture, and compressing the final mixture into a tablet.

In another embodiment, the method for manufacturing a pharmaceutical composition comprises the steps of forming a core structure comprising a first active agent formulated for immediate release and a second active agent formulated for extended release; coating the core structure with a delayed release coating to form a coated core structure; coating the coated core structure with a third active agent formulated for immediate release to form a double-coated core structure.

In some of the above-described methods for manufacturing a pharmaceutical composition, wherein the first and second active agents comprises acetaminophen and an NSAID, respectively, and at least one of the three active agents is selected from the group consisting of anti-obesity drugs, appetite suppressants, DGAT inhibitors, lipase inhibitors, and Type II diabetes agents.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the broadest possible scope consistent with the principles and features disclosed herein.

As used herein, the term “an effective amount” means an amount necessary to achieve a selected result.

As used herein, the term “nonsteroidal anti-inflammatory drug” or “NSAID” refers to a class of drugs that inhibit a cyclooxygenase enzyme, which does not include acetaminophen.

As used herein, the term “Type II diabetes agent” refers to a drug used to treat Type II diabetes.

As used herein, the term “weight-loss agent” refers to a drug that reduces weight or prevents the accumulation of weight.

The terms “treating” and “treatment” as used herein refer to reduction in body weight or prevention of weight gain. The phrase “administering to a subject” refers to the process of introducing a composition or dosage form of the invention into the subject (e.g., a human or other mammalian subject) via an art-recognized means of introduction.

The terms “effective amount” and “therapeutically effective amount” are used with reference to an agent, compound, drug, composition or combination of the invention which is nontoxic and effective for producing a desired therapeutic effect upon administration to a subject or patient (e.g., a human subject or patient).

As used herein, the term “analog” refers to a compound which comprises a chemically modified form of a specific compound or class thereof and which maintains the pharmaceutical and/or pharmacological activities characteristic of said compound or class.

As used herein, the term “derivative” refers to a chemically modified compound wherein the modification is considered routine by the ordinary skilled chemist, such as an ester or an amide of an acid, or protecting groups such as a benzyl group for an alcohol or thiol, or a tert-butoxycarbonyl group for an amine.

As used herein, the term “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric acid, hydrobromic acid, sulfuric acid, sulfamic acid, phosphoric acid, nitric acid, and the like and the salts prepared from organic acids such as acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluensulfonic acid, methanesulfonic acid, ethane dislfonic acid, oxalic acid, isethionic acid, and the like.

As used herein, the phrase “pharmaceutically acceptable” is used with reference to compounds, materials, compositions and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “immediate-release” is used herein with reference to a drug formulation that does not contain a dissolution rate controlling material. There is substantially no delay in the release of the active agents following administration of an immediate-release formulation. An immediate-release coating may include suitable materials immediately dissolving following administration so as to release the drug contents therein. In some embodiments, the term “immediate-release” is used with reference to a drug formulation that releases the active agent within two hours of administration.

As used herein, the term “extended-release,” also known as sustained-release (SR), sustained-action (SA), time-release (TR), controlled-release (CR), modified release (MR), or continuous-release (CR), refers to a mechanism used in medicine tablets or capsules to dissolve slowly and release the active agent over time, wherein the release is not immediate. The advantages of extended-release tablets or capsules are that they can often be taken less frequently than immediate-release formulations of the same drug and that they keep steadier levels of the drug in the bloodstream, thus extending the duration of the drug action and lowering the peak amount of drug in the bloodstream.

As used herein, the term “delayed-release” refers to a drug release profile that the release of the active agent(s) of a pharmaceutical composition is delayed or postponed for a given period of time (e.g., the lag period) after administration of the pharmaceutical composition. A formulation that releases its component “after gastric emptying” refers to a delayed formulation that releases the active agent(s) after the formulation is emptied from the stomach and enters intestine.

As used herein, the term “overweight” or the condition of having “excess weight” refers to a condition in which an individual has a body mass index (BMI) between 25.0 and 29.9 kg/m; the term “obesity” is defined as having a BMI of 30 or higher. BMI is calculated by dividing an individual's weight, in kilograms, by height, in meters.

One aspect of the present application relates to a method for reducing body weight by administering to a person in need thereof a pharmaceutical composition comprising acetaminophen in combination with a nonsteroidal anti-inflammatory drug (NSAID). The present method may be used for effecting weight loss and treating conditions associated with excess weight and obesity.

In another embodiment, the pharmaceutical composition further comprises one or more active agents selected from the group consisting of anti-obesity drugs, appetite suppressants, diacylglycerol acyltransferase (DGAT) inhibitors, lipase inhibitors, and Type II diabetes agents.

Exemplary NSAIDs include, but are not limited to, aceclofenac, acemetacin, acetaminophen, acetanilide, acetyl salicylic acid, alclofenac, alminoprofen, aminopyrine, ampiroxicam, amtolmetin, antipyrine, apazone, aspirin, azapropazone, benorylate, benoxaprofen, bromfenac, bucloxic acid, carprofen, celecoxib, choline magnesium trisalicylate, cizolirine, clidanac, clonixin, clopinac, coxibs, d-indobufen, dapsone, darbufelone, dexketoprofen, diclofenac, diflunisal, dipyrone, droxicam, etodolac, etoricoxib, fenamates, fenbufen, fenclofenec, fenoprofen, fentiazac, feprazone, floctafenine, flufenamic acid, flufenisal, fluprofen, flurbiprofen, furofenac, heteroaryl acetic acid derivatives, ibufenac, ibuprofen, indomethacin, indoprofen, isobutylphenyl propionic acid, isoxepac, isoxicam, ketoprofen, ketorolac, licofelone, lornoxicam, loxoprofen, lumiracoxib, meclofen, meclofenamate, meclofenamic acid, mefenamic, mefenamic acid, meloxicam, methyl salicylate, miroprofen, mofezolac, nabumetone, naproxen, naproxen sodium, niflumic acid, nimesulide, olsalazine, oxaprozin, oxaprozin; enolic acids, oxicam derivatives, oxipinac, oxyphenbutazone, para-aminophenol derivatives, parecoxib, phenacetin, phenylbutazone, piprofen, piroxicam, pirprofen, pivoxicam, podophyllotoxin derivatives, pranoprofen, prapoprofen, proglumetacin, propionic acid derivatives, pyrazolon derivatives, rofecoxib, salicylate, salicylates, salicylic acid, salsalate, sodium meclofenamate, sudoxicam, sulfasalazine, sulindac, suprofen, tenoxicam, tiaprofenic acid, tiopinac, tioxaprofen, tiracoxib, tolfenamic acid, tolmetin, valdecoxib, zaltoprofen, zidometacin, zomepirac, pharmacological salts thereof, hydrates thereof, and solvates thereof.

Anti-obesity drugs include bupropion/naltrexone (Contrave), liraglutide (Saxenda), lorcaserin (Belviq), orlistat (Xenical), and phentermine/topiramate (Qsymia), fluoxetine, bupropion, topiramate and ecopipam.

Appetite suppressants include aminorex, amphechloral, amphetamine, benzphetamine, caffeine, chlorphentermine, clobenzorex, cloforex, clominorex, clortermine, cyclexedrine, dexamphetamine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, leptin, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine.

Diacylglycerol acyltransferase (DGAT) inhibitors inhibit DGAT-catalyzed formation of triglycerides from diacylglycerol and Acyl-CoA. The reaction catalyzed by DGAT is considered the terminal and only committed step in triglyceride synthesis and to be essential for intestinal absorption (i.e. DGAT1) and adipose tissue formation (i.e. DGAT2). Exemplary DGAT inhibitors include pradigstat (LCQ-908), JTT-553, PF-04415060, and T863. Additional DGAT inhibitors are described in U.S. Pat. Nos. 9,340,566, 8,772,901 and 8,188,092.

Lipase inhibitors act locally in the intestinal tract to inhibit both gastric and pancreatic lipase. Exemplary lipase inhibitors include atglistatin, cetilistat, ebelactone, esterastin, hesperidin, lipstatin, orlistat, panclicin D, tetrahydrolipstatin, valilactone, and vibralactone.

Type 2 diabetes agents include, but are not limited to biguanides, DPP-IV (dipeptidyl peptidase-IV) inhibitors, glucagon-like peptide-1 (GLP-1) receptor agonists, α-glucosidase inhibitors, PPAR agonists, and sodium-glucose co-transporter 2 (SGLT-2) inhibitors.

Biguanides include metformin, buformin and phenformin.

DPP-IV inhibitors inhibit the DPP-IV enzyme and are used in the treatment of type 2 diabetes. Inhibition of the DPP-4 enzyme prolongs and enhances the activity of incretins, which play an important role in insulin secretion and blood glucose control regulation. DPP-IV inhibitors include alogliptin, evogliptin, gemigliptin, linagliptin, omarigliptin, saxagliptin, septagliptin, sitagliptin, teneligliptin, trelagliptin, and vildagliptin.

Glucagon-like peptide-1 (GLP-1) receptor agonists target the GLP-1 receptor and mimic the activity of incretin, which is released from the intestine in response to food intake. GLP-1 receptor agonists can increase insulin secretion, decrease glucagon release, increase satiety, and slow gastric emptying. GLP-1 receptor agonists include albiglutide, dulaglutide, exenatide, liraglutide, lixisenatide, semaglutide, and taspoglutide.

α-glucosidase inhibitors include voglibose, emiglitate, miglitol, and acarbose.

PPAR agonists act upon the peroxisome proliferator-activated receptor and are used for the treatment of symptoms of the metabolic syndrome, mainly for lowering triglycerides and blood sugar. The PPAR agonist can be a PPAR-alpha agonist, a PPAR-gamma agonist, or a dual PPAR-gamma/alpha agonist. PPAR-alpha agonists include bezafibrate, ciprofibrate, clofibrate, fenofibrate, and gemfibrozil. PPAR-alpha antagonists further include endocannabinoid pathway analogs, such as oleoylethanolamine (OEA), which regulates PPAR-α activity to stimulate lipolysis through the cannabinoid receptor GPR119. PPAR-gamma agonists include thiazolidinediones or glitazones, such as balaglitzaone, ciglitazone, darglitazone, lobeglitazone, netoglitazone, pioglitzone, rivoglitazone, rosiglitazone, and troglitazone. Dual PPAR-gamma/alpha agonists include aleglitazar, muraglitazar, and tesaglitazar.

SGLT-2 inhibitors include canagliflozin, dapagliflozin, empagliflozen, ipragliflozen, luseogliflozin, remoglifoxin, sergliflozin, and togogliflozin.

The compounds of this invention may be used in combination therapies for various indications, including but not limited to obesity, diseases associated with obesity, metabolic syndrome/diabesity, regulation of appetite and satiety, treatment of eating disorders, such as bulimia, maintenance of a reduced body weight following successful weight loss, reduction or prevention of hypoglycemia (both acute and chronic), Type 2 diabetes mellitus, delaying progression from prediabetes to type 2 diabetes, e.g. in states of impaired glucose tolerance and/or impaired fasting glucose, gestational diabetes, type 1 diabetes mellitus, reduction or prevention of hypoglycemia (both acute and chronic), and combinations thereof.

The active agent combinations can be used especially for a synergistic improvement in action. They can be applied either by separate administration of the active agents to the patient or in the form of combination products in which a plurality of active agents is present in one pharmaceutical preparation. When the active agents are administered by separate administration of the active agents, this can be done simultaneously or successively.

In another aspect, the pharmaceutical composition may be formulated for immediate-release, extended-release, delayed-release, or a combination thereof.

In one embodiment, the pharmaceutical composition is formulated for extended-release by embedding the active agent in a matrix of insoluble substance(s) such as acrylics or chitin. An extended-release form is designed to release the active agents at a predetermined rate by maintaining a constant drug level for a specific period of time. This can be achieved through a variety of formulations, including, but not limited to, liposomes and drug-polymer conjugates, such as hydrogels.

An extended-release formulation can be designed to release the active agents at a predetermined rate so as to maintain a constant drug level for a specified, extended period of time, such as up to about 12 hours, about 11 hours, about 10 hours, about 9 hours, about 8 hours, about 7 hours, about 6 hours, about 5 hours, about 4 hours, about 3 hours, about 2 hours, or about 1 hour following administration or following a lag period associated with delayed-release of the drug.

In certain embodiments, the active agents are released over a time interval of between about 2 to about 12 hours or about 1 to about 24 hours. Alternatively, the active agents may be released over about 3, about 4, about 5, about 6, about 7, about 8, about 9, about 10 hours, about 11 hours, or about 12 hours. In yet other embodiments, the active agents are released over a time period between about 5 to about 8 hours following administration.

In some embodiments, the extended-release formulation comprises an active core comprised of one or more inert particles, each in the form of a bead, pellet, pill, granular particle, microcapsule, microsphere, microgranule, nanocapsule, or nanosphere coated on its surfaces with drugs in the form of e.g., a drug-containing coating or film-forming composition using, for example, fluid bed techniques or other methodologies known to those of skill in the art. The inert particle can be of various sizes, so long as it is large enough to remain poorly dissolved. Alternatively, the active core may be prepared by granulating and milling and/or by extrusion and spheronization of a polymer composition containing the drug substance.

The active agents may be introduced to the inert carrier by techniques known to one skilled in the art, such as drug layering, powder coating, extrusion spheronization, roller compaction or granulation. The amount of drug in the core will depend on the dose that is required and typically varies from about 5 to 90 weight %. Generally, the polymeric coating on the active core will be from about 1 to 50% based on the weight of the coated particle, depending on the lag time required and/or the polymers and coating solvents chosen. Those skilled in the art will be able to select an appropriate amount of drug for coating onto or incorporating into the core to achieve the desired dosage. In one embodiment, the inactive core may be a sugar sphere or a buffer crystal or an encapsulated buffer crystal such as calcium carbonate, sodium bicarbonate, fumaric acid, tartaric acid, etc. which alters the microenvironment of the drug to facilitate its release.

Extended-release formulations may utilize a variety of extended-release coatings or mechanisms facilitating the gradual release of active agents over time. In some embodiments, the extended-release agent comprises a polymer controlling release by dissolution controlled release. In a particular embodiment, the active agent(s) is incorporated in a matrix comprising an insoluble polymer and drug particles or granules coated with polymeric materials of varying thickness. The polymeric material may comprise a lipid barrier comprising a waxy material, such as carnauba wax, beeswax, spermaceti wax, candellila wax, shallac wax, cocoa butter, cetostearyl alcohol, partially hydrogenated vegetable oils, ceresin, paraffin wax, ceresine, myristyl alcohol, stearyl alcohol, cetyl alcohol, and stearic acid, along with surfactants, such as polyoxyethylene sorbitan monooleate. When contacted with an aqueous medium, such as biological fluids, the polymer coating emulsifies or erodes after a predetermined lag-time depending on the thickness of the polymer coating. The lag time is independent of gastrointestinal motility, pH, or gastric residence. In some embodiments, the extended-release formulation is in the form of an orally disintegrating tablet, a powder or a liquid suspension. One or more of the active agents, however, are present in coated particles or are embedded in particles that slowly release the active agent(s) over the desired period of time.

In other embodiments, the extended-release agent comprises a polymeric matrix effecting diffusion controlled release. The matrix may comprise one or more hydrophilic and/or water-swellable, matrix forming polymers, pH-dependent polymers and/or pH-independent polymers.

In one embodiment, the extended-release formulation comprises a water soluble or water-swellable matrix-forming polymer, optionally containing one or more solubility-enhancing agents and/or release-promoting agents. Upon solubilization of the water soluble polymer, the active agent(s) dissolves (if soluble) and gradually diffuses through the hydrated portion of the matrix. The gel layer grows with time as more water permeates into the core of the matrix, increasing the thickness of the gel layer and providing a diffusion barrier to drug release. As the outer layer becomes fully hydrated, the polymer chains become completely relaxed and can no longer maintain the integrity of the gel layer, leading to disentanglement and erosion of the outer hydrated polymer on the surface of the matrix. Water continues to penetrate towards the core through the gel layer, until it has been completely eroded. Whereas soluble drugs are released by this combination of diffusion and erosion mechanisms, erosion is the predominant mechanism for insoluble drugs, regardless of dose.

Similarly, water-swellable polymers typically hydrate and swell in biological fluids forming a homogenous matrix structure that maintains its shape during drug release and serves as a carrier for the drug, solubility enhancers and/or release promoters. The initial matrix polymer hydration phase results in slow-release of the drug (lag phase). Once the water swellable polymer is fully hydrated and swollen, water within the matrix can similarly dissolve the drug substance and allow for its diffusion out through the matrix coating.

Additionally, the porosity of the matrix can be increased due to the leaching out of pH-dependent release promoters so as to release the drug at a faster rate. The rate of the drug release then becomes constant and is a function of drug diffusion through the hydrated polymer gel. The release rate from the matrix is dependent upon various factors, including polymer type and level, drug solubility and dose, polymer to drug ratio, filler type and level, polymer to filler ratio, particle size of drug and polymer, and porosity and shape of the matrix.

Exemplary hydrophilic and/or water-swellable, matrix forming polymers include, but are not limited to, cellulosic polymers including hydroxyalkyl celluloses and carboxyalkyl celluloses such as hydroxypropylmethylcellulose (HPMC), hydroxypropylcellulose (HPC), hydroxyethylcellulose (HEC), methylcellulose (MC), carboxymethylcellulose (CMC); powdered cellulose such as microcrystalline cellulose, cellulose acetate, ethylcellulose, salts thereof, and combinations thereof; alginates; gums including heteropolysaccharide gums and homopolysaccharide gums such as xanthan, tragacanth, pectin, acacia, karaya, alginates, agar, guar, hydroxypropyl guar, veegum, carrageenan, locust bean gum, gellan gum, and derivatives therefrom; acrylic resins including polymers and copolymers of acrylic acid, methacrylic acid, methyl acrylate, and methyl methacrylate; and cross-linked polyacrylic acid derivatives such as Carbomers (e.g., CARBOPOL®, including CARBOPOL® 71G NF, which is available in various molecular weight grades from Noveon, Inc., Cincinnati, Ohio); carageenan; polyvinyl acetate (e.g., KOLLIDON® SR); and polyvinyl pyrrolidone and its derivatives such as crospovidone, polyethylene oxides, and polyvinyl alcohol. Preferred hydrophilic and water-swellable polymers include the cellulosic polymers, especially HPMC.

The extended-release formulation may further comprise at least one binder that is capable of cross-linking the hydrophilic compound to form a hydrophilic polymer matrix (i.e., a gel matrix) in an aqueous medium, including biological fluids.

Exemplary binders include homopolysaccharides, such as galactomannan gums, guar gum, hydroxypropyl guar gum, hydroxypropylcellulose (HPC; e.g., Klucel EXF), and locust bean gum. In other embodiments, the binder is an alginic acid derivative, HPC or microcrystallized cellulose (MCC). Other binders include, but are not limited to, starches, microcrystalline cellulose, hydroxypropyl cellulose, hydroxyethyl cellulose, hydroxypropylmethyl cellulose, and polyvinylpyrrolidone.

In one embodiment, the introduction method is drug layering by spraying a suspension of active agent(s) and a binder onto the inert carrier.

The binder may be present in the bead formulation in an amount of from about 0.1% to about 15% by weight and preferably of from about 0.2% to about 10% by weight.

In some embodiments, the hydrophilic polymer matrix may further include an ionic polymer, a non-ionic polymer, or water-insoluble hydrophobic polymer to provide a stronger gel layer and/or reduce pore quantity and dimensions in the matrix so as to slow diffusion and erosion rates and concomitant release of the active agent(s). This may additionally suppress the initial burst effect and produce a more steady, “zero order release” of active agent(s).

Exemplary ionic polymers for slowing dissolution rate include both anionic and cationic polymers. Exemplary anionic polymers include, for example, sodium carboxymethylcellulose (Na CMC); sodium alginate; polymers of acrylic acid or carbomers (e.g., CARBOPOL® 934, 940, 974P NF); enteric polymers such as polyvinyl acetate phthalate (PVAP), methacrylic acid copolymers (e.g., EUDRAGIT® L100, L 30D 55, A, and FS 30D), and hypromellose acetate succinate (AQUAT HPMCAS); and xanthan gum. Exemplary cationic polymers include, for example, dimethylaminoethyl methacrylate copolymer (e.g., EUDRAGIT® E 100). Incorporation of anionic polymers, particularly enteric polymers, is useful for developing a pH-independent release profile for weakly basic drugs as compared to hydrophilic polymer alone.

Exemplary non-ionic polymers for slowing dissolution rate, include, for example, hydroxypropylcellulose (HPC) and polyethylene oxide (PEO) (e.g., POLYOX™)

Exemplary hydrophobic polymers include ethylcellulose (e.g., ETHOCEL™, SURELEASE®), cellulose acetate, methacrylic acid copolymers (e.g., EUDRAGIT® NE 30D), ammonio-methacrylate copolymers (e.g., EUDRAGIT® RL 100 or PO RS100), polyvinyl acetate, glyceryl monostearate, fatty acids such as acetyl tributyl citrate, and combinations and derivatives thereof.

The swellable polymer can be incorporated in the formulation in proportion from 1% to 50% by weight, preferably from 5% to 40% by weight, most preferably from 5% to 20% by weight. The swellable polymers and binders may be incorporated in the formulation either prior to or after granulation. The polymers can also be dispersed in organic solvents or hydro-alcohols and sprayed during granulation.

Exemplary release-promoting agents include pH-dependent enteric polymers that remain intact at pH value lower than about 4.0 and dissolve at pH values higher than 4.0, preferably higher than 5.0, most preferably about 6.0, are considered useful as release-promoting agents for this invention. Exemplary pH-dependent polymers include, but are not limited to, methacarylic acid copolymers; methacrylic acid-methyl methacrylate copolymers (e.g., EUDRAGIT® L100 (Type A), EUDRAGIT® 5100 (Type B), Rohm GmbH, Germany); methacrylic acid-ethyl acrylate copolymers (e.g., EUDRAGIT® L100-55 (Type C) and EUDRAGIT® L30D-55 copolymer dispersion, Rohm GmbH, Germany); copolymers of methacrylic acid-methyl methacrylate and methyl methacrylate (EUDRAGIT® FS); terpolymers of methacrylic acid, methacrylate, and ethyl acrylate; cellulose acetate phthalates (CAP); hydroxypropyl methylcellulose phthalate (HPMCP) (e.g., HP-55, HP-50, HP-55S, Shinetsu Chemical, Japan); polyvinyl acetate phthalates (PVAP) (e.g., COATERIC®, OPADRY® enteric white OY-P-7171); polyvinylbutyrate acetate; cellulose acetate succinates (CAS); hydroxypropyl methylcellulose acetate succinate (HPMCAS) (e.g., HPMCAS LF Grade, MF Grade, and HF Grade, including AQOAT® LF and AQOAT® MF, Shin-Etsu Chemical, Japan); shellac (e.g., MARCOAT™ 125 and MARCOAT™ 125N); vinyl acetate-maleic anhydride copolymer; styrene-maleic monoester copolymer; carboxymethyl ethylcellulose (CMEC, Freund Corporation, Japan); cellulose acetate phthalates (CAP) (e.g., AQUATERIC®); cellulose acetate trimellitates (CAT); and mixtures of two or more thereof at weight ratios between about 2:1 to about 5:1, such as a mixture of EUDRAGIT® L 100-55 and EUDRAGIT® S 100 at a weight ratio of about 3:1 to about 2:1 or a mixture of EUDRAGIT® L 30 D-55 and EUDRAGIT® FS at a weight ratio of about 3:1 to about 5:1.

These polymers may be used either alone or in combination, or together with polymers other than those mentioned above. Preferred enteric pH-dependent polymers are the pharmaceutically acceptable methacrylic acid copolymers. These copolymers are anionic polymers based on methacrylic acid and methyl methacrylate and, preferably, have a mean molecular weight of about 135,000. A ratio of free carboxyl groups to methyl-esterified carboxyl groups in these copolymers may range, for example, from 1:1 to 1:3, e.g. around 1:1 or 1:2. Such polymers are sold under the trade name Eudragit® such as the Eudragit L series e.g., Eudragit L 12.5®, Eudragit L 12.5P®, Eudragit L100®, Eudragit L 100-55®, Eudragit L-30D®, Eudragit L-30 D-55®, the Eudragit S® series e.g., Eudragit S 12.5®, Eudragit S 12.5P®, Eudragit S100®. The release promoters are not limited to pH dependent polymers. Other hydrophilic molecules that dissolve rapidly and leach out of the dosage form quickly leaving a porous structure can be also be used for the same purpose.

In some embodiments, the matrix may include a combination of release promoters and solubility enhancing agents. The solubility enhancing agents can be ionic and non-ionic surfactants, complexing agents, hydrophilic polymers, and pH modifiers such as acidifying agents and alkalinizing agents, as well as molecules that increase the solubility of poorly soluble drug through molecular entrapment. Several solubility enhancing agents can be utilized simultaneously.

Solubility enhancing agents may include surface active agents, such as sodium docusate; sodium lauryl sulfate; sodium stearyl fumarate; Tweens® and Spans (PEO modified sorbitan monoesters and fatty acid sorbitan esters); poly(ethylene oxide)-polypropylene oxide-poly(ethylene oxide) block copolymers (aka PLURONICS™); complexing agents such as low molecular weight polyvinyl pyrrolidone and low molecular weight hydroxypropyl methyl cellulose; molecules that aid solubility by molecular entrapment such as cyclodextrins and pH modifying agents, including acidifying agents such as citric acid, fumaric acid, tartaric acid, and hydrochloric acid; and alkalizing agents such as meglumine and sodium hydroxide.

Solubility enhancing agents typically constitute from 1% to 80% by weight, preferably from 1% to 60%, more preferably from 1% to 50%, of the dosage form and can be incorporated in a variety of ways. They can be incorporated in the formulation prior to granulation in dry or wet form. They can also be added to the formulation after the rest of the materials are granulated or otherwise processed. During granulation, solubility enhancing agents can be sprayed as solutions with or without a binder.

In one embodiment, the extended-release formulation comprises a water-insoluble water-permeable polymeric coating or matrix comprising one or more water-insoluble water-permeable film-forming over the active core. The coating may additionally include one or more water soluble polymers and/or one or more plasticizers. The water-insoluble polymer coating comprises a barrier coating for release of active agents in the core, wherein lower molecular weight (viscosity) grades exhibit faster release rates as compared to higher viscosity grades.

In some embodiments, the water-insoluble film-forming polymers include one or more alkyl cellulose ethers, such as ethyl celluloses and mixtures thereof, (e.g., ethyl cellulose grades PR100, PR45, PR20, PR10, and PR7; ETHOCEL®, Dow).

In some embodiments, the water-insoluble polymer provides suitable properties (e.g., extended-release characteristics, mechanical properties, and coating properties) without the need for a plasticizer. For example, coatings comprising polyvinyl acetate (PVA), neutral copolymers of acrylate/methacrylate esters such as commercially available Eudragit NE30D from Evonik Industries, ethyl cellulose in combination with hydroxypropylcellulose, waxes, etc. can be applied without plasticizers.

In yet another embodiment, the water-insoluble polymer matrix may further include a plasticizer. The amount of plasticizer required depends upon the plasticizer, the properties of the water-insoluble polymer and the ultimate desired properties of the coating. Suitable levels of plasticizer range from about 1% to about 20%, from about 3% to about 20%, about 3% to about 5%, about 7% to about 10%, about 12% to about 15%, about 17% to about 20%, or about 1%, about 2%, about 3%, about 4%, about 5%, about 6%, about 7%, about 8%, about 9%, about 10%, about 15%, or about 20% by weight relative to the total weight of the coating, inclusive of all ranges and sub-ranges therebetween.

Exemplary plasticizers include, but are not limited to, triacetin, acetylated monoglyceride, oils (castor oil, hydrogenated castor oil, grape seed oil, sesame oil, olive oil, and etc.), citrate esters, triethyl citrate, acetyltriethyl citrate acetyltributyl citrate, tributyl citrate, acetyl tri-n-butyl citrate, diethyl phthalate, dibutyl phthalate, dioctyl phthalate, methyl paraben, propyl paraben, propyl paraben, butyl paraben, diethyl sebacate, dibutyl sebacate, glyceroltributyrate, substituted triglycerides and glycerides, monoacetylated and diacetylated glycerides (e.g., MYVACET® 9-45), glyceryl monostearate, glycerol tributyrate, polysorbate 80, polyethyleneglycol (such as PEG-4000 and PEG-400), propyleneglycol, 1,2-propyleneglycol, glycerin, sorbitol, diethyl oxalate, diethyl malate, diethyl fumarate, diethylmalonate, dibutyl succinate, fatty acids, glycerin, sorbitol, diethyl oxalate, diethyl malate, diethyl maleate, diethyl fumarate, diethyl succinate, diethyl malonate, dioctyl phthalate, dibutyl sebacate, and mixtures thereof. The plasticizer can have surfactant properties, such that it can act as a release modifier. For example, non-ionic detergents such as Brij 58 (polyoxyethylene (20) cetyl ether), and the like, can be used.

Plasticizers can be high boiling point organic solvents used to impart flexibility to otherwise hard or brittle polymeric materials and can affect the release profile for the active agent(s). Plasticizers generally cause a reduction in the cohesive intermolecular forces along the polymer chains resulting in various changes in polymer properties including a reduction in tensile strength and increase in elongation and a reduction in the glass transition or softening temperature of the polymer. The amount and choice of the plasticizer can affect the hardness of a tablet, for example, and can even affect its dissolution or disintegration characteristics, as well as its physical and chemical stability. Certain plasticizers can increase the elasticity and/or pliability of a coat, thereby decreasing the coat's brittleness.

In another embodiment, the extended-release formulation comprises a combination of at least two gel-forming polymers, including at least one non-ionic gel-forming polymer and/or at least one anionic gel-forming polymer. The gel formed by the combination of gel-forming polymers provides controlled release, such that when the formulation is ingested and comes into contact with the gastrointestinal fluids, the polymers nearest the surface hydrate to form a viscous gel layer. Because of the high viscosity, the viscous layer dissolves away only gradually, exposing the material below to the same process. The mass thus dissolves away slowly, thereby slowly releasing the active agent into the gastrointestinal fluids. The combination of at least two gel-forming polymers enables properties of the resultant gel, such as viscosity, to be manipulated in order to provide the desired release profile.

In a particular embodiment, the formulation comprises at least one non-ionic gel-forming polymer and at least one anionic gel-forming polymer. In another embodiment, the formulation comprises two different non-ionic gel-forming polymers. In yet another embodiment, the formulation comprises a combination of non-ionic gel-forming polymers with the same chemistry, but solubilities, viscosities, and/or molecular weights (for example a combination of hydroxyproplyl methylcellulose of different viscosity grades, such as HPMC K100 and HPMC K15M or HPMC K100M).

Exemplary anionic gel forming polymers include, but are not limited to, sodium carboxymethylcellulose (Na CMC), carboxymethyl cellulose (CMC), anionic polysaccharides such as sodium alginate, alginic acid, pectin, polyglucuronic acid (poly-α- and -β-1,4-glucuronic acid), polygalacturonic acid (pectic acid), chondroitin sulfate, carrageenan, furcellaran, anionic gums such as xanthan gum, polymers of acrylic acid or carbomers (Carbopol® 934, 940, 974P NF), Carbopol® copolymers, a Pemulen® polymer, polycarbophil, and others.

Exemplary non-ionic gel-forming polymers include, but are not limited to, Povidone (PVP: polyvinyl pyrrolidone), polyvinyl alcohol, copolymer of PVP and polyvinyl acetate, HPC (hydroxypropyl cellulose), HPMC (hydroxypropyl methylcellulose), hydroxyethyl cellulose, hydroxymethyl cellulose, gelatin, polyethylene oxide, acacia, dextrin, starch, polyhydroxyethylmethacrylate (PHEMA), water soluble nonionic polymethacrylates and their copolymers, modified cellulose, modified polysaccharides, nonionic gums, nonionic polysaccharides, and/or mixtures thereof.

The formulation may optionally comprise an enteric polymer as described above and/or at least one excipient, such as a filler, a binder (as described above), a disintegrant and/or a flow aid or glidant.

Exemplary fillers include but are not limited to, lactose; glucose; fructose; sucrose; dicalcium phosphate; sugar alcohols also known as “sugar polyol” such as sorbitol, manitol, lactitol, xylitol, isomalt, erythritol, and hydrogenated starch hydrolysates (a blend of several sugar alcohols); corn starch; potato starch; sodium carboxymethycellulose; ethylcellulose and cellulose acetate; enteric polymers; or a mixture thereof.

Exemplary binders include, but are not limited to, water-soluble hydrophilic polymers such as Povidone (PVP: polyvinyl pyrrolidone), copovidone (a copolymer of polyvinyl pyrrolidone and polyvinyl acetate), low molecular weight HPC (hydroxypropyl cellulose), low molecular weight HPMC (hydroxypropyl methylcellulose), low molecular weight carboxy methyl cellulose, ethylcellulose, gelatin, polyethylene oxide, acacia, dextrin, magnesium aluminum silicate, and starch and polymethacrylates such as Eudragit NE 30D, Eudragit RL, Eudragit RS, Eudragit E, polyvinyl acetate, enteric polymers, or mixtures thereof.

Exemplary disintegrants include, but are not limited to, low-substituted carboxymethyl cellulose sodium, crospovidone (cross-linked polyvinyl pyrrolidone), sodium carboxymethyl starch (sodium starch glycolate), cross-linked sodium carboxymethyl cellulose (Croscarmellose), pregelatinized starch (starch 1500), microcrystalline cellulose, water insoluble starch, calcium carboxymethyl cellulose, low substituted hydroxypropyl cellulose, and magnesium or aluminum silicate.

Exemplary glidants include but are not limited to, magnesium, silicon dioxide, talc, starch, titanium dioxide, and the like.

In yet another embodiment, the extended-release formulation is formed by coating a water soluble/dispersible drug-containing particle, such as a bead or bead population therein (as described above), with a coating material and, optionally, a pore former and other excipients. The coating material is preferably selected from a group comprising cellulosic polymers such as ethylcellulose (e.g., SURELEASE®), methylcellulose, hydroxypropyl cellulose, hydroxypropylmethyl cellulose, cellulose acetate, and cellulose acetate phthalate; polyvinyl alcohol; acrylic polymers such as polyacrylates, polymethacrylates, and copolymers thereof; and other water-based or solvent-based coating materials. The release-controlling coating for a given bead population may be controlled by at least one parameter of the release controlling coating, such as the nature of the coating, coating level, type and concentration of a pore former, process parameters, and combinations thereof. Thus, changing a parameter, such as a pore former concentration, or the conditions of the curing, allows for changes in the release of active agent(s) from any given bead population, thereby allowing for selective adjustment of the formulation to a pre-determined release profile.

Pore formers suitable for use in the release controlling coating herein can be organic or inorganic agents and include materials that can be dissolved, extracted or leached from the coating in the environment of use. Exemplary pore forming agents include, but are not limited to, organic compounds such as mono-, oligo-, and polysaccharides including sucrose, glucose, fructose, mannitol, mannose, galactose, sorbitol, pullulan, and dextran; polymers soluble in the environment of use such as water-soluble hydrophilic polymers, hydroxyalkylcelluloses, carboxyalkylcelluloses, hydroxypropylmethylcellulose, cellulose ethers, acrylic resins, polyvinylpyrrolidone, cross-linked polyvinylpyrrolidone, polyethylene oxide, Carbowaxes, Carbopol, and the like, diols, polyols, polyhydric alcohols, polyalkylene glycols, polyethylene glycols, polypropylene glycols, or block polymers thereof, polyglycols, and poly(α-Ω)alkylenediols; and inorganic compounds such as alkali metal salts, lithium carbonate, sodium chloride, sodium bromide, potassium chloride, potassium sulfate, potassium phosphate, sodium acetate, sodium citrate, suitable calcium salts, combination thereof, and the like.

The release controlling coating can further comprise other additives known in the art, such as plasticizers, anti-adherents, glidants (or flow aids), and antifoams.

In some embodiments, the coated particles or beads may additionally include an “overcoat,” to provide, e.g., moisture protection, static charge reduction, taste-masking, flavoring, coloring, and/or polish or other cosmetic appeal to the beads. Suitable coating materials for such an overcoat are known in the art and include, but are not limited to, cellulosic polymers such as hydroxypropylmethylcellulose, hydroxypropylcellulose, and microcrystalline cellulose or combinations thereof (for example, various OPADRY® coating materials).

The coated particles or beads may additionally contain enhancers that may be exemplified by, but not limited to, solubility enhancers, dissolution enhancers, absorption enhancers, permeability enhancers, stabilizers, complexing agents, enzyme inhibitors, p-glycoprotein inhibitors, and multidrug resistance protein inhibitors. Alternatively, the formulation can also contain enhancers that are separated from the coated particles, for example in a separate population of beads or as a powder. In yet another embodiment, the enhancer(s) may be contained in a separate layer on coated particles either under or above the release controlling coating.

In other embodiments, the extended-release formulation is formulated to release the active agent(s) by an osmotic mechanism. By way of example, a capsule may be formulated with a single osmotic unit or it may incorporate 2, 3, 4, 5, or 6 push-pull units encapsulated within a hard gelatin capsule, whereby each bilayer push pull unit contains an osmotic push layer and a drug layer, both surrounded by a semi-permeable membrane. One or more orifices are drilled through the membrane next to the drug layer. This membrane may be additionally covered with a pH-dependent enteric coating to prevent release until after gastric emptying. The gelatin capsule dissolves immediately after ingestion. As the push pull unit(s) enters the small intestine, the enteric coating breaks down, which then allows fluid to flow through the semi-permeable membrane, swelling the osmotic push compartment to force to force drugs out through the orifice(s) at a rate precisely controlled by the rate of water transport through the semi-permeable membrane. Release of drugs can occur over a constant rate for up to 24 hours or more.

The osmotic push layer comprises one or more osmotic agents creating the driving force for transport of water through the semi-permeable membrane into the core of the delivery vehicle. One class of osmotic agents includes water-swellable hydrophilic polymers, also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

Another class of osmotic agents includes osmogens, which are capable of imbibing water to effect an osmotic pressure gradient across the semi-permeable membrane. Exemplary osmogens include, but are not limited to, inorganic salts such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars such as dextrose, fructose, glucose, inositol, lactose, maltose, mannitol, raffinose, sorbitol, sucrose, trehalose, and xylitol; organic acids such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking.

In some embodiments, the extended-release formulation comprises a polysaccharide coating that is resistant to erosion in both the stomach and intestine. Such polymers can be only degraded in the colon, which contains a large microflora containing biodegradable enzymes breaking down, for example, the polysaccharide coatings to release the drug contents in a controlled, time-dependent manner. Exemplary polysaccharide coatings may include, for example, amylose, arabinogalactan, chitosan, chondroitin sulfate, cyclodextrin, dextran, guar gum, pectin, xylan, and combinations or derivatives therefrom.

In some embodiments, the pharmaceutical composition is formulated for delayed extended-release. As used herein, the term “delayed extended-release” is used with reference to a drug formulation having a release profile in which there is a predetermined delay in the release of the drug following administration and, once initiated, the drug is released continuously over an extended period of time. In some embodiments, the delayed extended-release formulation includes an extended-release formulation coated with an enteric coating, which is a barrier applied to oral medication that prevents release of medication before it reaches the small intestine. Delayed-release formulations, such as enteric coatings, prevent drugs having an irritant effect on the stomach, such as aspirin, from dissolving in the stomach. Such coatings are also used to protect acid-unstable drugs from the stomach's acidic exposure, delivering them instead to a basic pH environment (intestine's pH 5.5 and above) where they do not degrade and give their desired action. The term “pulsatile release” is a type of delayed-release, which is used herein with reference to a drug formulation that provides rapid and transient release of the drug within a short time period immediately after a predetermined lag period, thereby producing a “pulsed” plasma profile of the drug after drug administration. Formulations may be designed to provide a single pulsatile release or multiple pulsatile releases at predetermined time intervals following administration, or a pulsatile release (e.g., 20-60% of the active agent) followed with extended release over a period of time (e.g., a continuous release of the remainder of the active agent). A delayed-release or pulsatile release formulation generally comprises one or more elements covered with a barrier coating, which dissolves, erodes or ruptures following a specified lag phase.

A barrier coating for delayed-release may consist of a variety of different materials, depending on the objective. In addition, a formulation may comprise a plurality of barrier coatings to facilitate release in a temporal manner. The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols, and/or polyvinylpyrrolidone) or a coating based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, the formulation may additionally include a time delay material such as, for example, glyceryl monostearate or glyceryl distearate.

In some embodiments, the delayed, extended-release formulation includes an enteric coating comprised one or more polymers facilitating release of active agents in proximal or distal regions of the gastrointestinal tract. As used herein, the term “enteric coating” is a coating comprising of one or more polymers having a pH dependent or pH-independent release profile. An enteric coated pill will not dissolve in the acidic juices of the stomach (pH^{˜}3), but they will in the alkaline (pH 7-9) environment present in the small intestine or colon. An enteric polymer coating typically resists releases of the active agents until sometime after a gastric emptying lag period of about 3-4 hours after administration.

pH dependent enteric coatings comprise one or more pH-dependent or pH-sensitive polymers that maintain their structural integrity at low pH, as in the stomach, but dissolve in higher pH environments in more distal regions of the gastrointestinal tract, such as the small intestine, where the drug contents are released. For purposes of the present invention, “pH dependent” is defined as having characteristics (e.g., dissolution) which vary according to environmental pH. Exemplary pH-dependent polymers have been described earlier. pH-dependent polymers typically exhibit a characteristic pH optimum for dissolution. In some embodiments, the pH-dependent polymer exhibits a pH optimum between about 5.0 and 5.5, between about 5.5 and 6.0, between about 6.0 and 6.5, or between about 6.5 and 7.0. In other embodiments, the pH-dependent polymer exhibits a pH optimum of ≥5.0, of ≥5.5, odf ≥6.0, of ≥6.5, or of ≥7.0.

In certain embodiments, the coating methodology employs the blending of one or more pH-dependent and one or more pH-independent polymers. The blending of pH-dependent and pH-independent polymers can reduce the release rate of active agents once the soluble polymer has reached its optimum pH of solubilization.

In some embodiments, a “time-controlled” or “time-dependent” release profile can be obtained using a water insoluble capsule body containing one or more active agents, wherein the capsule body closed at one end with an insoluble, but permeable and swellable hydrogel plug. Upon contact with gastrointestinal fluid or dissolution medium, the plug swells, pushing itself out of the capsule and releasing the drugs after a pre-determined lag time, which can be controlled by e.g., the position and dimensions of the plug. The capsule body may be further coated with an outer pH-dependent enteric coating keeping the capsule intact until it reaches the small intestine. Suitable plug materials include, for example, polymethacrylates, erodible compressed polymers (e.g., HPMC, polyvinyl alcohol), congealed melted polymer (e.g., glyceryl mono oleate), and enzymatically controlled erodible polymers (e.g., polysaccharides, such as amylose, arabinogalactan, chitosan, chondroitin sulfate, cyclodextrin, dextran, guar gum, pectin and xylan).

In other embodiments, capsules or bilayered tablets may be formulated to contain a drug-containing core, covered by a swelling layer and an outer insoluble, but semi-permeable polymer coating or membrane. The lag time prior to rupture can be controlled by the permeation and mechanical properties of the polymer coating and the swelling behavior of the swelling layer. Typically, the swelling layer comprises one or more swelling agents, such as swellable hydrophilic polymers that swell and retain water in their structures.

Exemplary water swellable materials to be used in the delayed-release coating include, but are not limited to, polyethylene oxides (having e.g., an average molecular weight between 1,000,000 and 7,000,000, such as POLYOX®); methylcellulose; hydroxypropyl cellulose; hydroxypropyl methylcellulose; polyalkylene oxides having a weight average molecular weight of 100,000 to 6,000,000, including, but not limited to, poly(methylene oxide), poly(butylene oxide), poly(hydroxy alkyl methacrylate) having a molecular weight of 25,000 to 5,000,000, poly(vinyl)alcohol having a low acetal residue, which is cross-linked with glyoxal, formaldehyde, or glutaraldehyde, and having a degree of polymerization from 200 to 30,000; mixtures of methyl cellulose, cross-linked agar, and carboxymethyl cellulose; hydrogel forming copolymers produced by forming a dispersion of a finely divided copolymer of maleic anhydride with styrene, ethylene, propylene, butylene, or isobutylene cross-linked with from 0.001 to 0.5 moles of saturated cross-linking agent per mole of maleic anyhydride in the copolymer; CARBOPOL® acidic carboxy polymers having a molecular weight of 450,000 to 4,000,000; CYANAMER® polyacrylamides; cross-linked water swellable indenemaleicanhydride polymers; GOODRITE® polyacrylic acid having a molecular weight of 80,000 to 200,000; starch graft copolymers; AQUA-KEEPS® acrylate polymer polysaccharides composed of condensed glucose units such as diester cross-linked polyglucan; carbomers having a viscosity of 3,000 to 60,000 mPa as a 0.5%-1% w/v aqueous solution; cellulose ethers such as hydroxypropylcellulose having a viscosity of about 1000-7000 mPa s as a 1% w/w aqueous solution (25° C.); hydroxypropyl methylcellulose having a viscosity of about 1000 or higher, preferably 2,500 or higher to a maximum of 25,000 mPa as a 2% w/v aqueous solution; polyvinylpyrrolidone having a viscosity of about 300-700 mPa s as a 10% w/v aqueous solution at 20° C.; and combinations thereof.

Alternatively, the release time of the drugs can be controlled by a disintegration lag time depending on the balance between the tolerability and thickness of a water insoluble polymer membrane (such as ethyl cellulose, EC) containing predefined micropores at the bottom of the body and the amount of a swellable excipient, such as low substituted hydroxypropyl cellulose (L-HPC) and sodium glycolate. After oral administration, GI fluids permeate through the micropores, causing swelling of the swellable excipients, which produces an inner pressure disengaging the capsular components, including a first capsule body containing the swellable materials, a second capsule body containing the drugs, and an outer cap attached to the first capsule body.

The enteric layer may further comprise anti-tackiness agents, such as talc or glyceryl monostearate and/or plasticizers. The enteric layer may further comprise one or more plasticizers including, but not limited to, triethyl citrate, acetyl triethyl citrate, acetyltributyl citrate, polyethylene glycol acetylated monoglycerides, glycerin, triacetin, propylene glycol, phthalate esters (e.g., diethyl phthalate, dibutyl phthalate), titanium dioxide, ferric oxides, castor oil, sorbitol, and dibutyl sebacate.

In another embodiment, the delayed release formulation employs a water-permeable but insoluble film coating to enclose the active agent and an osmotic agent. As water from the gut slowly diffuses through the film into the core, the core swells until the film bursts, thereby releasing the active agents. The film coating may be adjusted to permit various rates of water permeation or release time.

In another embodiment, the delayed release formulation employs a water-impermeable tablet coating whereby water enters through a controlled aperture in the coating until the core bursts. When the tablet bursts, the drug contents are released immediately or over a longer period of time. These and other techniques may be modified to allow for a pre-determined lag period before release of drugs is initiated.

In another embodiment, the active agents are delivered in a formulation to provide both delayed-release and extended-release (delayed-extended-release). The term “delayed-extended-release” is used herein with reference to a drug formulation providing pulsatile release of active agents at a pre-determined time or lag period following administration, which is then followed by extended-release of the active agents thereafter.

In some embodiments, immediate-release, extended-release, delayed-release, or delayed-extended-release formulations comprises an active core comprised of one or more inert particles, each in the form of a bead, pellet, pill, granular particle, microcapsule, microsphere, microgranule, nanocapsule, or nanosphere coated on its surfaces with drugs in the form of e.g., a drug-containing film-forming composition using, for example, fluid bed techniques or other methodologies known to those of skill in the art. The inert particle can be of various sizes, so long as it is large enough to remain poorly dissolved. Alternatively, the active core may be prepared by granulating and milling and/or by extrusion and spheronization of a polymer composition containing the drug substance.

The amount of drug in the core will depend on the dose that is required and typically varies from about 5 to 90 weight %. Generally, the polymeric coating on the active core will be from about 1 to 50% based on the weight of the coated particle, depending on the lag time and type of release profile required and/or the polymers and coating solvents chosen. Those skilled in the art will be able to select an appropriate amount of drug for coating onto or incorporating into the core to achieve the desired dosage. In one embodiment, the inactive core may be a sugar sphere or a buffer crystal or an encapsulated buffer crystal such as calcium carbonate, sodium bicarbonate, fumaric acid, tartaric acid, etc. which alters the microenvironment of the drug to facilitate its release.

In some embodiments, for example, delayed-release or delayed-extended-release compositions may formed by coating a water soluble/dispersible drug-containing particle, such as a bead, with a mixture of a water insoluble polymer and an enteric polymer, wherein the water insoluble polymer and the enteric polymer may be present at a weight ratio of from 4:1 to 1:1, and the total weight of the coatings is 10 to 60 weight % based on the total weight of the coated beads. The drug layered beads may optionally include an inner dissolution rate controlling membrane of ethylcellulose. The composition of the outer layer, as well as the individual weights of the inner and outer layers of the polymeric membrane are optimized for achieving desired circadian rhythm release profiles for a given active, which are predicted based on in vitro/in vivo correlations.

In other embodiments the formulations may comprise a mixture of immediate-release drug-containing particles without a dissolution rate controlling polymer membrane and delayed-extended-release beads exhibiting, for example, a lag time of 2-4 hours following oral administration, thus providing a two-pulse release profile.

In some embodiments, the active core is coated with one or more layers of dissolution rate-controlling polymers to obtain desired release profiles with or without a lag time. An inner layer membrane can largely control the rate of drug release following imbibition of water or body fluids into the core, while the outer layer membrane can provide for a desired lag time (the period of no or little drug release following imbibition of water or body fluids into the core). The inner layer membrane may comprise a water insoluble polymer, or a mixture of water insoluble and water soluble polymers.

The polymers suitable for the outer membrane, which largely controls the lag time of up to 6 hours may comprise an enteric polymer, as described above, and a water insoluble polymer at 10 to 50 weight %. The ratio of water insoluble polymer to enteric polymer may vary from 4:1 to 1:2, preferably the polymers are present at a ratio of about 1:1. The water insoluble polymer typically used is ethylcellulose.

Exemplary water insoluble polymers include ethylcellulose, polyvinyl acetate (Kollicoat SR#0D from BASF), neutral copolymers based on ethyl acrylate and methylmethacrylate, copolymers of acrylic and methacrylic acid esters with quaternary ammonium groups such as EUDRAGIT® NE, RS and RS30D, RL or RL30D, and the like. Exemplary water soluble polymers include low molecular weight HPMC, HPC, methylcellulose, polyethylene glycol (PEG of molecular weight>3000) at a thickness ranging from 1 weight % up to 10 weight % depending on the solubility of the active in water and the solvent or latex suspension based coating formulation used. The water insoluble polymer to water soluble polymer may typically vary from 95:5 to 60:40, preferably from 80:20 to 65:35. In some embodiments, AMBERLITE™ IRP69 resin is used as an extended-release carrier. AMBERLITE™ IRP69 is an insoluble, strongly acidic, sodium form cation exchange resin that is suitable as carrier for cationic (basic) substances. In other embodiments, DUOLITE™ AP143/1093 resin is used as an extended-release carrier. DUOLITE™ AP143/1093 is an insoluble, strongly basic, anion exchange resin that is suitable as a carrier for anionic (acidic) substances. When used as a drug carrier, AMBERLITE™ IRP69 or/and DUOLITE™ AP143/1093 resin provides a means for binding medicinal agents onto an insoluble polymeric matrix. Extended-release is achieved through the formation of resin-drug complexes (drug resinates). The drug is released from the resin in vivo as the drug reaches equilibrium with the high electrolyte concentrations, which are typical of the gastrointestinal tract. More hydrophobic drugs will usually elute from the resin at a lower rate, owing to hydrophobic interactions with the aromatic structure of the cation exchange system.

In some embodiments, the pharmaceutical composition is formulated for oral administration. Oral dosage forms include, for example, tablets, capsules, and caplets and may also comprise a plurality of granules, beads, powders, or pellets that may or may not be encapsulated. Tablets and capsules represent the most convenient oral dosage forms, in which case solid pharmaceutical carriers are employed.

In a delayed-release formulation, one or more barrier coatings may be applied to pellets, tablets, or capsules to facilitate slow dissolution and concomitant release of drugs into the intestine. Typically, the barrier coating contains one or more polymers encasing, surrounding, or forming a layer, or membrane around the therapeutic composition or active core. In some embodiments, the active agents are delivered in a formulation to provide delayed-release at a pre-determined time following administration. The delay may be up to about 10 minutes, about 20 minutes, about 30 minutes, about 1 hour, about 2 hours, about 3 hours, about 4 hours, about 5 hours, about 6 hours, or longer.

Various coating techniques may be applied to granules, beads, powders or pellets, tablets, capsules or combinations thereof containing active agents to produce different and distinct release profiles. In some embodiments, the pharmaceutical composition is in a tablet or capsule form containing a single coating layer. In other embodiments, the pharmaceutical composition is in a tablet or capsule form containing multiple coating layers. In some embodiments, the pharmaceutical composition of the present application is formulated for extended-release or delayed extended-release of up to 100% of the active agent.

In other embodiments, the pharmaceutical composition of the present application is formulated for a two-phase extended-release or delayed two-phase extended-release characterized by an “immediate-release” component that is released within two hours of administration and an “extended-release” component which is released over a period of 2-12 hours. In some embodiments, the “immediate-release” component provides about 20-60% of the total dosage of the active agent(s) and the “extended-release” component provides 40-80% of the total dosage of the active agent(s) to be delivered by the pharmaceutical formulation. For example, the immediate-release component may provide about 20-60%, or about 20%, 25%, 30%, 35%, 40%, 45%, 50%, 55%, 60% of the total dosage of the active agent(s) to be delivered by the pharmaceutical formulation. The extended-release component provides about 40%, 45%, 50%, 55%, 60%, 65%, 70%, 75% or 80% of the total dosage of the active agent(s) to be delivered by the formulation.

In some embodiments, the immediate-release component and the extended-release component contain the same active agent(s). In other embodiments, the immediate-release component and the extended-release component contain different active agents (e.g., acetaminophen in one component and an NSAID in another component).

In some embodiments, the immediate-release component and the extended-release component each contains acetaminophen and at least one NSAID. In other embodiments, the immediate-release component and/or the extended-release component further comprises one or more additional active agents selected from the group consisting of anti-obesity drugs, appetite suppressants, diacylglycerol acyltransferase (DGAT) inhibitors, lipase inhibitors, and Type II diabetes agents.

In some embodiments, the pharmaceutical composition further comprises a plurality of active agents selected from the group consisting of anti-obesity drugs, appetite suppressants, diacylglycerol acyltransferase (DGAT) inhibitors, lipase inhibitors, and Type II diabetes agents.

In some embodiments, the pharmaceutical composition comprises (1) acetaminophen, (2) an NSAID, and (3) one or more active agents selected from the group consisting of anti-obesity drugs, appetite suppressants, diacylglycerol acyltransferase (DGAT) inhibitors, lipase inhibitors, Type II diabetes agents, and combinations thereof.

In one embodiment, the pharmaceutical composition comprises (1) acetaminophen; (2) an NSAID, and (3) an anti-obesity drug. In another embodiment, the pharmaceutical composition comprises (1) acetaminophen; (2) an NSAID, and (3) an appetite suppressant. In another embodiment, the pharmaceutical composition comprises (1) acetaminophen; (2) an NSAID, and (3) a DGAT inhibitor. In another embodiment, the pharmaceutical composition comprises (1) acetaminophen; (2) an NSAID, and (3) a lipase inhibitor. In another embodiment, the pharmaceutical composition comprises (1) acetaminophen; (2) an NSAID, and (3) a Type II diabetes agent.

In another embodiment, the pharmaceutical composition comprises (1) acetaminophen; (2) an NSAID, and (3) at least two different Type II diabetes agents selected from the group consisting of biguanides, DPP-IV inhibitors, glucagon-like peptide-1 receptor agonists, α-glucosidase inhibitors, PPAR agonists, and SGLT-2 inhibitors.

In another embodiment, the pharmaceutical composition comprises (1) acetaminophen; (2) an NSAID, (3) an anti-obesity drug; and (4) an appetite suppressant. In another embodiment, the pharmaceutical composition comprises (1) acetaminophen; (2) an NSAID, (3) an anti-obesity drug; and (4) an appetite suppressant. In another embodiment, the pharmaceutical composition comprises (1) acetaminophen; (2) an NSAID, (3) an anti-obesity drug; and (4) a DGAT inhibitor. In another embodiment, the pharmaceutical composition comprises (1) acetaminophen; (2) an NSAID, (3) an anti-obesity drug; and (4) a lipase inhibitor. In another embodiment, the pharmaceutical composition comprises (1) acetaminophen; (2) an NSAID, (3) an appetite suppressant; and (4) a DGAT inhibitor. In another embodiment, the pharmaceutical composition comprises (1) acetaminophen; (2) an NSAID, (3) an appetite suppressant; and (4) a lipase inhibitor. In another embodiment, the pharmaceutical composition comprises (1) acetaminophen; (2) an NSAID; (3) a DGAT inhibitor; and (4) a lipase inhibitor. In another embodiment, the pharmaceutical composition comprises (1) acetaminophen; (2) an NSAID, (3) a DGAT inhibitor; and (4) a Type II diabetes agent. In another embodiment, the pharmaceutical composition comprises (1) acetaminophen; (2) an NSAID, (3) a lipase inhibitor; and (4) a Type II diabetes agent.

In one embodiment, the plurality of active agents is formulated for immediate-release. In other embodiment, the plurality of active agents is formulated for extended-release. In other embodiment, the plurality of active agents is formulated for both immediate-release and extended-release (e.g., a first portion of each active agent is formulated for immediate-release and a second portion of each active agent is formulated for extended-release). In yet other embodiment, some of the plurality of active agents are formulated for immediate-release and some of the plurality of active agents are formulated for extended-release (e.g., active agents A, B, C are formulated for immediate-release and active agents C and D are formulated for extended-release). In some other embodiments, the immediate-release component and/or the extended-release component is further coated with a delayed-release coating, such as an enteric coating.

In certain embodiments, the pharmaceutical composition comprises an immediate-release component and an extended-release component. Each of the immediate-release component and/or the extended release may comprise one or more active agents selected from the group consisting of acetaminophen, NSAIDs, anti-obesity drugs, appetite suppressants, diacylglycerol acyltransferase (DGAT) inhibitors, lipase inhibitors, Type II diabetes agents, and combinations thereof. In some embodiments, the immediate-release component and the extended-release component have exactly the same active agents. In other embodiments, the immediate-release component and the extended-release component have different active agents. In yet other embodiments, the immediate-release component and the extended-release component have one or more common active agents. In some other embodiments, the immediate-release component and/or the extended-release component is further coated with a delayed-release coating, such as an enteric coating.

In one embodiment, the pharmaceutical composition comprises a plurality of active agents in which one or more of the active agents are formulated for immediate-release at about the same time. In another embodiment, the pharmaceutical composition comprises a plurality of active agents in which one or more of the active agents are formulated for extended-release at about the same time. In another embodiment, the pharmaceutical composition comprises at least two or more active agents formulated as two extended-release components, each providing a different extended-release profile. For example, a first extended-release component releases a first active agent at a first release rate and a second extended-release component releases a second active agent at a second release rate. In another embodiment, the pharmaceutical composition comprises at least two or more active agents, both formulated for delayed release.

In another embodiment, the pharmaceutical composition comprises one or more active agents formulated for delayed release. In another embodiment, the pharmaceutical composition comprises at least two or more active agents formulated as two delayed-release components, each providing a different delayed-release profile. For example, a first delayed-release component releases a first active agent at a first time point, and a second delayed-release component releases a second active agent at a second time point.

In other embodiments, the pharmaceutical composition comprises acetaminophen and/or an NSAID formulated for immediate-release and (2) one or more active agents selected from the group consisting of anti-obesity drugs, appetite suppressants, diacylglycerol acyltransferase (DGAT) inhibitors, lipase inhibitors, Type II diabetes agents, and combinations thereof, which is/are formulated for extended-release. In other embodiments, the pharmaceutical composition comprises three active agents formulated for immediate-release and three active agents formulated for extended-release. In other embodiments, the pharmaceutical composition comprises four active agents formulated for immediate-release and (2) four active agents formulated for extended-release. In these embodiments, the active agent(s) in the immediate-release component can be the same as, or different from, the active agent(s) in the extended-release component. In other embodiments, the immediate-release component and/or the extended-release component is further coated with a delayed-release coating, such as an enteric coating. The delayed-release coating may be formulated to delay the release of the active agent for a period of 1, 2, 3, 4 or 5 hours.

An immediate-release composition may comprise 100% of the total dosage of a given active agent administered in a single unit dose. Alternatively, an immediate-release component may be included as a component in a combined release profile formulation that may provide about 1% to about 60% of the total dosage of the active agent(s) to be delivered by the pharmaceutical formulation. For example, the immediate-release component may provide about 5%-60%, about 10% to about 60%, about 10% to about 50%, about 10% to about 40%, about 10% to about 30%, about 10% to about 20%, about 20% to about 60%, about 20% to about 50%, about 20% to about 30%, about 30% to about 60%, about 30% to about 50%, about 40% to about 60%, about 40% to about 50%, about 45% to about 60% or about 45% to about 50% of the total dosage of the active agent(s) to be delivered by the formulation. In alternate embodiments, the immediate-release component provides about 2, 4, 5, 10, 15, 20, 25, 30, 35, 40, 45, 50, 55 or 60% of the total dosage of the active agent(s) to be delivered by the formulation.

In some embodiments, the immediate-release or delayed-release formulation comprises an active core comprised of one or more inert particles, each in the form of a bead, pellet, pill, granular particle, microcapsule, microsphere, microgranule, nanocapsule, or nanosphere coated on its surfaces with drugs in the form of e.g., a drug-containing film-forming composition using, for example, fluid bed techniques or other methodologies known to those of skill in the art. The inert particle can be of various sizes, so long as it is large enough to remain poorly dissolved. Alternatively, the active core may be prepared by granulating and milling and/or by extrusion and spheronization of a polymer composition containing the drug substance.

The amount of drug in the core will depend on the dose that is required and typically varies from about 5 to 90 weight %. Generally, the polymeric coating on the active core will be from about 1 to 50% based on the weight of the coated particle, depending on the lag time and type of release profile required and/or the polymers and coating solvents chosen. Those skilled in the art will be able to select an appropriate amount of drug for coating onto or incorporating into the core to achieve the desired dosage. In one embodiment, the inactive core may be a sugar sphere or a buffer crystal or an encapsulated buffer crystal such as calcium carbonate, sodium bicarbonate, fumaric acid, tartaric acid, etc. which alters the microenvironment of the drug to facilitate its release.

In some embodiments, the delayed-release formulation is formed by coating a water soluble/dispersible drug-containing particle, such as a bead, with a mixture of a water insoluble polymer and an enteric polymer, wherein the water insoluble polymer and the enteric polymer may be present at a weight ratio of 4:1 to 1:1, and the total weight of the coatings is 10 to 60 weight % based on the total weight of the coated beads. The drug layered beads may optionally include an inner dissolution rate controlling membrane of ethylcellulose. The composition of the outer layer, as well as the individual weights of the inner and outer layers of the polymeric membrane are optimized for achieving desired circadian rhythm release profiles for a given active, which are predicted based on in vitro/in vivo correlations.

In other embodiments the formulations comprise a mixture of immediate-release drug-containing particles without a dissolution rate controlling polymer membrane and delayed release beads exhibiting, for example, a lag time of 2-4 hours following oral administration, thus providing a two-pulse release profile. In yet other embodiments the formulations comprise a mixture of two types of delayed-release beads: a first type that exhibits a lag time of 1-3 hours and a second type that exhibits a lag time of 4-6 hours. In yet other embodiments the formulations comprise a mixture of two types of release beads: a first type that exhibits immediate-release and a second type that exhibits a lag time of 1-4 hours followed with extended-release.

In other embodiments, the formulations are designed with a release profile such that a fraction of the medicine (e.g., 20-60%) is released immediately or within two hours of administration, and the rest is released over an extended period of time. The pharmaceutical composition may be administered daily or administered on an as needed basis. In certain embodiments, the pharmaceutical composition is administered to the subject prior to bedtime. In some embodiments, the pharmaceutical composition is administered immediately before bedtime. In some embodiments, the pharmaceutical composition is administered within about two hours before bedtime, preferably within about one hour before bedtime. In another embodiment, the pharmaceutical composition is administered about two hours before bedtime. In a further embodiment, the pharmaceutical composition is administered at least two hours before bedtime. In another embodiment, the pharmaceutical composition is administered about one hour before bedtime. In a further embodiment, the pharmaceutical composition is administered at least one hour before bedtime. In still another embodiment, the pharmaceutical composition is administered immediately before bedtime. Preferably, the pharmaceutical composition is administered orally.

The appropriate dosage (“therapeutically effective amount”) of the active agent(s) in the immediate-release component, the extended-release component, the delayed-release component or delayed-extended-release component will depend, for example, on the severity and course of the condition, the mode of administration, the bioavailability of the particular agent(s), the age and weight of the patient, the patient's clinical history and response to the active agent(s), discretion of the physician, etc.

As a general proposition, the therapeutically effective amount of the active agent(s) in the immediate-release component, the delayed-release component, the extended-release component or the delayed-extended-release component is administered in a range from about 10 μg/kg body weight/day to about 100 mg/kg body weight/day, whether by one or more administrations. In some embodiments, the range of each active agent administered daily in a single dose or in multiple does is from about 10 μg/kg body weight/day to about 100 mg/kg body weight/day, 10 μg/kg body weight/day to about 30 mg/kg body weight/day, 10 μg/kg body weight/day to about 10 mg/kg body weight/day, 10 μg/kg body weight/day to about 3 mg/kg body weight/day, 10 μg/kg body weight/day to about 1 mg/kg body weight/day, 10 μg/kg body weight/day to about 300 μg/kg body weight/day, 10 μg/kg body weight/day to about 100 μg/kg body weight/day, 10 μg/kg body weight/day to about 30 μg/kg body weight/day, 30 μg/kg body weight/day to about 100 mg/kg body weight/day, 30 μg/kg body weight/day to about 30 mg/kg body weight/day, 30 μg/kg body weight/day to about 10 mg/kg body weight/day, 30 μg/kg body weight/day to about 3 mg/kg body weight/day, 30 μg/kg body weight/day to about 1 mg/kg body weight/day, 30 μg/kg body weight/day to about 300 μg/kg body weight/day, 30 μg/kg body weight/day to about 100 μg/kg body weight/day, 100 μg/kg body weight/day to about 100 mg/kg body weight/day, 100 μg/kg body weight/day to about 30 mg/kg body weight/day, 100 μg/kg body weight/day to about 10 mg/kg body weight/day, 100 μg/kg body weight/day to about 3 mg/kg body weight/day, 100 μg/kg body weight/day to about 1 mg/kg body weight/day, 100 μg/kg body weight/day to about 300 μg/kg body weight/day, 300 μg/kg body weight/day to about 100 mg/kg body weight/day, 300 μg/kg body weight/day to about 30 mg/kg body weight/day, 300 μg/kg body weight/day to about 10 mg/kg body weight/day, 300 μg/kg body weight/day to about 3 mg/kg body weight/day, 300 μg/kg body weight/day to about 1 mg/kg body weight/day, 1 mg/kg body weight/day to about 100 mg/kg body weight/day, 1 mg/kg body weight/day to about 30 mg/kg body weight/day, 1 mg/kg body weight/day to about 10 mg/kg body weight/day, 1 mg/kg body weight/day to about 3 mg/kg body weight/day, 3 mg/kg body weight/day to about 100 mg/kg body weight/day, 3 mg/kg body weight/day to about 30 mg/kg body weight/day, 3 mg/kg body weight/day to about 10 mg/kg body weight/day, 10 mg/kg body weight/day to about 100 mg/kg body weight/day, 10 mg/kg body weight/day to about 30 mg/kg body weight/day or 30 mg/kg body weight/day to about 100 mg/kg body weight/day.

The active agent(s) described herein may be included in an immediate-release component or an extended-release component, a delayed-release component, a delayed-extended-release component or combinations thereof for daily oral administration at a single dose or combined dose range of 1 mg to 2000 mg, 1 mg to 1000 mg, 1 mg to 300 mg, 1 mg to 100 mg, 1 mg to 30 mg, 1 mg to 10 mg, 1 mg to 3 mg, 3 mg to 2000 mg, 3 mg to 1000 mg, 3 mg to 300 mg, 3 mg to 100 mg, 3 mg to 30 mg, 3 mg to 10 mg, 10 mg to 2000 mg, 10 mg to 1000 mg, 10 mg to 300 mg, 10 mg to 100 mg, 10 mg to 30 mg, 30 mg to 2000 mg, 30 mg to 1000 mg, 30 mg to 300 mg, 30 mg to 100 mg, 100 mg to 2000 mg, 100 mg to 1000 mg, 100 mg to 300 mg, 300 mg to 2000 mg, 300 mg to 1000 mg or 1000 mg to 2000 mg. As expected, the dosage will be dependent on the condition, size, age, and condition of the patient.

In some embodiments, the pharmaceutical composition comprises acetaminophen in combination with a single NSAID, a single anti-obesity drug, a single appetite suppressant, a single DGAT inhibitor, a single lipase inhibitor, a single Type II diabetes agent, or any combination thereof. In a preferred embodiment, the NSAID is ibuprofen. In some embodiments, the pharmaceutical composition comprises a plurality of different Type II diabetes agents selected from the group consisting of biguanides, DPP-IV inhibitors, glucagon-like peptide-1 receptor agonists, α-glucosidase inhibitors, PPAR agonists, and SGLT-2 inhibitors.

Acetaminophen may be combined with one or more NSAIDS in a weight ratio in the range of 0.1:1 to 10:1, 0.2:1 to 5:1 or 0.3:1 to 3:1. In one embodiment, acetaminophen is combined with an NSAID at a weight ratio in the range of 2:1 to 3:1.

In some other embodiments, the pharmaceutical composition of the present application further comprises one or more anti-obesity drugs. Examples of the anti-obesity drugs include, but are not limited to, bupropion/naltrexone (Contrave), liraglutide (Saxenda), lorcaserin (Belviq), orlistat (Xenical), and phentermine/topiramate (Qsymia), fluoxetine, bupropion, topiramate or ecopipam. The daily dose for each of the one or more anti-obesity drug(s) may be in the range of 1 μg to 300 mg, 1 μg to 100 mg, 1 μg to 30 mg; 1 μg to 10 mg, 1 μg to 3 mg, 1 μg to 1 mg, 1 μg to 300 μg to 100 μg, 1 μg to 30 μg, 1 μg to 10 μg, 1 μg to 3 μg, 3 μg to 100 mg, 3 μg to 100 mg, 3 μg to 30 mg; 3 μg to 10 mg, 3 μg to 3 mg, 3 μg to 1 mg, 3 μg to 300 μg, 3 μg to 100 μg, 3 μg to 30 μg, 3 μg to 10 μg, 10 μg to 300 mg, 10 μg to 100 mg, 10 μg to 30 mg; 10 μg to 10 mg, 10 μg to 3 mg, 10 μg to 1 mg, 10 μg to 300 μg, 10 μg to 100 μg, 10 μg to 30 μg, 30 μg to 300 mg, 30 μg to 100 mg, 30 μg to 30 mg; 30 μg to 10 mg, 30 μg to 3 mg, 30 μg to 1 mg, 30 μg to 300 30 μg to 100 100 μg to 300 mg, 100 μg to 100 mg, 100 μg to 30 mg; 100 μg to 10 mg, 100 μg to 3 mg, 100 μg to 1 mg, 100 μg to 300 μg, 300 μg to 300 mg, 300 μg to 100 mg, 300 μg to 30 mg; 300 μg to 10 mg, 300 μg to 3 mg, 300 μg to 1 mg, 1 mg to 300 mg, 1 mμg to 100 mg, 1 mg to 30 mg, 1 mg to 3 mg, 3 mg to 300 mg, 3 mg to 100 mg, 3 mg to 30 mg, 3 mg to 10 mg, 10 mg to 300 mg, 10 mg to 100 mg, 10 mg to 30 mg, 30 mg to 300 mg, 30 mg to 100 mg or 100 mg to 300 mg.

In some other embodiments, the pharmaceutical composition of the present application further comprises one or more appetite suppressants. Examples of appetite suppressants include, but are not limited to, include aminorex, amphechloral, amphetamine, benzphetamine, caffeine, chlorphentermine, clobenzorex, cloforex, clominorex, clortermine, cyclexedrine, dexamphetamine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, leptin, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine, and analogs, salts, hydrates, and solvates thereof. The daily dose for each the one or more appetite suppressant(s) may be in the range of 1 μg to 300 mg, 1 μg to 100 mg, 1 μg to 30 mg; 1 μg to 10 mg, 1 μg to 3 mg, 1 μg to 1 mg, 1 μg to 300 μg, 1 μg to 100 μg, 1 μg to 30 μg, 1 μg to 10 μg, 1 μg to 3 μg, 3 μg to 100 mg, 3 μg to 100 mg, 3 μg to 30 mg; 3 μg to 10 mg, 3 μg to 3 mg, 3 μg to 1 mg, 3 μg to 300 μg, 3 μg to 100 μg, 3 μg to 30 μg, 3 μg to 10 μg, 10 μg to 300 mg, 10 μg to 100 mg, 10 μg to 30 mg; 10 μg to 10 mg, 10 μg to 3 mg, 10 μg to 1 mg, 10 μg to 300 μg, 10 μg to 100 μg, 10 μg to 30 μg, 30 μg to 300 mg, 30 μg to 100 mg, 30 μg to 30 mg; 30 μg to 10 mg, 30 μg to 3 mg, 30 μg to 1 mg, 30 μg to 300 μg, 30 μg to 100 μg, 100 μg to 300 mg, 100 μg to 100 mg, 100 μg to 30 mg; 100 μg to 10 mg, 100 μg to 3 mg, 100 μg to 1 mg, 100 μg to 300 μg, 300 μg to 300 mg, 300 μg to 100 mg, 300 μg to 30 mg; 300 μg to 10 mg, 300 μg to 3 mg, 300 μg to 1 mg, 1 mg to 300 mg, 1 mg to 100 mg, 1 mg to 30 mg, 1 mg to 3 mg, 3 mg to 300 mg, 3 mg to 100 mg, 3 mg to 30 mg, 3 mg to 10 mg, 10 mg to 300 mg, 10 mg to 100 mg, 10 mg to 30 mg, 30 mg to 300 mg, 30 mg to 100 mg or 100 mg to 300 mg.

In other embodiments, the pharmaceutical composition of the present application further comprises one or more DGAT inhibitors. Examples of DGAT inhibitors include, but are not limited to, pradigstat (LCQ-908), JTT-553, PF-04415060, and T863. Each of the one or more DGAT inhibitor(s) may be used at a daily dose of 0.1 mg to 1000 mg, 0.1 mg to 300 mg, 0.1 mg to 100 mg, 0.1 mg to 30 mg, 0.1 mg to 10 mg, 0.1 mg to 3 mg, 0.1 mg to 1 mg, 0.1 mg to 0.3 mg, 0.3 mg to 1000 mg, 0.3 mg to 300 mg, 0.3 mg to 100 mg, 0.3 mg to 30 mg, 0.3 mg to 10 mg, 0.3 mg to 3 mg, 0.3 mg to 1 mg, 1 mg to 1000 mg, 1 mg to 300 mg, 1 mg to 100 mg, 1 mg to 30 mg, 1 mg to 10 mg, 1 mg to 3 mg, 3 mg to 1000 mg, 3 mg to 300 mg, 3 mg to 100 mg, 3 mg to 30 mg, 3 mg to 10 mg, 10 mg to 1000 mg, 10 mg to 300 mg, 10 mg to 100 mg, 10 mg to 30 mg, 30 mg to 1000 mg, 30 mg to 300 mg, 30 mg to 100 mg, 100 mg to 1000 mg, 100 mg to 300 mg, or 300 mg to 1000 mg.

In other embodiments, the pharmaceutical composition of the present application further comprises one or more lipase inhibitors. Exemplary lipase inhibitors include, but are not limited to, atglistatin, cetilistat, ebelactone, esterastin, hesperidin, lipstatin, orlistat, panclicin D, tetrahydrolipstatin, valilactone, and vibralactone. Each of the one or more lipase inhibitor(s) may be used at a daily dose of 0.1 mg to 1000 mg, 0.1 mg to 300 mg, 0.1 mg to 100 mg, 0.1 mg to 30 mg, 0.1 mg to 10 mg, 0.1 mg to 3 mg, 0.1 mg to 1 mg, 0.1 mg to 0.3 mg, 0.3 mg to 1000 mg, 0.3 mg to 300 mg, 0.3 mg to 100 mg, 0.3 mg to 30 mg, 0.3 mg to 10 mg, 0.3 mg to 3 mg, 0.3 mg to 1 mg, 1 mg to 1000 mg, 1 mg to 300 mg, 1 mg to 100 mg, 1 mg to 30 mg, 1 mg to 10 mg, 1 mg to 3 mg, 3 mg to 1000 mg, 3 mg to 300 mg, 3 mg to 100 mg, 3 mg to 30 mg, 3 mg to 10 mg, 10 mg to 1000 mg, 10 mg to 300 mg, 10 mg to 100 mg, 10 mg to 30 mg, 30 mg to 1000 mg, 30 mg to 300 mg, 30 mg to 100 mg, 100 mg to 1000 mg, 100 mg to 300 mg, or 300 mg to 1000 mg.

In other embodiments, the pharmaceutical composition of the present application further comprises one or more Type 2 diabetes agents. The Type 2 diabetes agents may be selected from the group consisting of biguanides, DPP-IV (dipeptidyl peptidase-IV) inhibitors, glucagon-like peptide-1 (GLP-1) receptor agonists, α-glucosidase inhibitors, PPAR agonists, and sodium-glucose co-transporter 2 (SGLT-2) inhibitors. Exemplary biguanides include, but are not limited to, metformin, buformin and phenformin. Exemplary DPP-IV inhibitors include, but are not limited to, alogliptin, evogliptin, gemigliptin, linagliptin, omarigliptin, saxagliptin, septagliptin, sitagliptin, teneligliptin, trelagliptin, and vildagliptin. Exemplary GLP-1 receptor agonists include, but are not limited to, albiglutide, dulaglutide, exenatide, liraglutide, lixisenatide, semaglutide, and taspoglutide. Exemplary α-glucosidase inhibitors include, but are not limited to, voglibose, emiglitate, miglitol, and acarbose. Exemplary PPAR agonists include, but are not limited to, PPAR-alpha agonists, such as bezafibrate, ciprofibrate, clofibrate, fenofibrate, gemfibrozil, and oleoylethanolamine (OEA); PPAR-gamma agonists, such as thiazolidinediones or glitazones, such as balaglitzaone, ciglitazone, darglitazone, lobeglitazone, netoglitazone, pioglitzone, rivoglitazone, rosiglitazone, and troglitazone; and PPAR-gamma/alpha agonists, such as aleglitazar, muraglitazar, and tesaglitazar. Exemplary SGLT-2 inhibitors include, but are not limited to canagliflozin, dapagliflozin, empagliflozen, ipragliflozen, luseogliflozin, remoglifoxin, sergliflozin, and togogliflozin. Each of the one or more Type II diabetes agent(s) may be used at a daily dose of 0.1 mg to 1000 mg, 0.1 mg to 300 mg, 0.1 mg to 100 mg, 0.1 mg to 30 mg, 0.1 mg to 10 mg, 0.1 mg to 3 mg, 0.1 mg to 1 mg, 0.1 mg to 0.3 mg, 0.3 mg to 1000 mg, 0.3 mg to 300 mg, 0.3 mg to 100 mg, 0.3 mg to 30 mg, 0.3 mg to 10 mg, 0.3 mg to 3 mg, 0.3 mg to 1 mg, 1 mg to 1000 mg, 1 mg to 300 mg, 1 mg to 100 mg, 1 mg to 30 mg, 1 mg to 10 mg, 1 mg to 3 mg, 3 mg to 1000 mg, 3 mg to 300 mg, 3 mg to 100 mg, 3 mg to 30 mg, 3 mg to 10 mg, 10 mg to 1000 mg, 10 mg to 300 mg, 10 mg to 100 mg, 10 mg to 30 mg, 30 mg to 1000 mg, 30 mg to 300 mg, 30 mg to 100 mg, 100 mg to 1000 mg, 100 mg to 300 mg, or 300 mg to 1000 mg.

Each of the anti-obesity drugs, appetite suppressants, diacylglycerol acyltransferase (DGAT) inhibitors, lipase inhibitors, and/or Type II diabetes agents in the pharmaceutical composition may be formulated for immediate-release, extended-release, delayed release, delayed-extended-release or combinations thereof.

In some embodiments, the pharmaceutical composition of the present application further comprises an effective amount of an anti-insomnia agent. As used herein, the term “anti-insomnia agent” refers to agents, compounds or drugs used to treat insomnia. Exemplary anti-insomnia agents include, but are not limited to, barbiturates such as secobarbital, amobarbital, butalbital, cyclobarbital, pentobarbital, allobarbital, barbital, butobarbital, mephobarbital, phenobarbital, butabarbital, and vinylbital; quinazolinones and derivatives such as cloroqualone, diproqualone, etaqualone (Aolan, Athinazone, Ethinazone), mebroqualone, mecloqualone (Nubarene, Casfen), and methaqualone (Quaalude); benzodiazepines such as clorazepate, diazepam, flurazepam, halazepam, prazepam, lorazepam, lormetazepam, oxazepam, temazepam, clonazepam, flunitrazepam, nimetazepam, nitrazepam, adinazolam, alprazolam, estazolam, triazolam, climazolam, loprazolam, midazolam; non-benzodiazepines, such as zaleplon and eszopiclone; antihistamines such as diphenhydramine and doxylamine; melatonin; antidepressants such as trazodonee, amitriptyline, doxepin, trimipramine, mianserin and mirtazapine; antipsychotics such as chlorpromazine, clozapine, olanzapine, quetiapine, risperidone, zotepine, clonidine, guanfacine, cannabidiol, tetrahydrocannabinol, suvorexant, pregabalin, gabapentin, phenibut, imagabalin. In one embodiment, the pharmaceutical composition of the present application further comprises an effective amount of melatonin.

The pharmaceutical composition may be formulated into a tablet, an orally disintegrating tablet, capsule, dragee, powder, granulate, liquid, gel or emulsion form. Said liquid, gel or emulsion may be ingested by the subject in naked form or contained within a capsule.

In some embodiments, the composition is formulated for extended release with a release profile in which the acetaminophen and the one or more NSAIDs are released at a steady rate over a period of 2-12 hours or 5-8 hours. In some embodiments, the composition is formulated for extended release with a release profile in which at least 90% of the acetaminophen and the one or more NSAIDs are released continuously over a period of 2-12 hours or 5-8 hours. In another embodiment, the pharmaceutical composition is formulated for extended release, characterized by a two-phase release profile in which 20, 30, 40, 50 or 60% of the active agents are released within two hours of administration and remainder of the active agents are released continuously over a period of 2-12 hours. In a related embodiment, the pharmaceutical composition is coated with an enteric coating.

In other embodiments, the composition is formulated for extended release with a release profile in which the acetaminophen and the one or more NSAIDs are released continuously, or at a steady rate, over a period of 5, 6, 7, 8, 10 or 12 hours. As used herein, “a steady rate over a period of time” is defined as a release profile in which the release rate at any point during a given period of time is within 30%-300% of the average release rate over that given period of time. For example, if 80 mg of acetaminophen is released at a steady rate over a period of 8 hours, the average release rate is 10 mg/hr during this period of time and the actual release rate at any time during this period is within the range of 3 mg/hr to 30 mg/hr (i.e., within 30%-300% of the average release rate of 10 mg/hr during the 8 hour period). In some embodiments, these pharmaceutical compositions further comprise one or more active agents selected from the group consisting of anti-obesity drugs, appetite suppressants, diacylglycerol acyltransferase (DGAT) inhibitors, lipase inhibitors, and Type II diabetes agents.

In one embodiment, the pharmaceutical composition is formulated for extended release and comprises (1) acetaminophen; (2) one or more NSAIDs; and optionally (3) one or more active agents selected from the group consisting of anti-obesity drugs, appetite suppressants, diacylglycerol acyltransferase (DGAT) inhibitors, lipase inhibitors, Type II diabetes agents, anti-insomnia agents, wherein each active agent is present in the composition in an amount between 1-1000 mg, 1-800 mg, 1-600 mg, 1-500 mg, 1-400 mg, 1-300 mg, 1-250 mg, 1-200 mg, 1-150 mg, 1-100 mg, 1-50 mg, 1-20, 1-10 mg, 10-1000 mg, 10-800 mg, 10-600 mg, 10-500 mg, 10-400 mg, 10-300 mg, 10-250 mg, 10-200 mg, 10-150 mg, 10-100 mg, 30-1000 mg, 30-800 mg, 30-600 mg, 30-500 mg, 30-400 mg, 30-300 mg, 30-250 mg, 30-200 mg, 30-150 mg, 30-100 mg, 100-1000 mg, 100-800 mg, 100-600 mg, 100-400 mg, 100-250 mg, 300-1000 mg, 300-800 mg, 300-600 mg, 300-400 mg, 400-1000 mg, 400-800 mg, 400-600 mg, 600-1000 mg, 600-800 mg or 800-1000 mg, wherein the composition is formulated for extended release with a release profile in which the one or more active agent(s) are released continuously, or at a steady rate, over a period of 2-12 hours or 5-8 hours. Alternatively, or in addition, the acetaminophen and the one or more NSAIDs may be formulated for extended release with a two-phase release profile in which 20, 30, 40, 50 or 60% of the active agent(s) are released within 2 hours of administration and the remainder are released continuously, or at a steady rate, over a period of 2-12 hours or 5-8 hours.

The pharmaceutical composition is formulated to provide a steady release of acetaminophen and the one or more NSAIDs to maintain an effective drug concentration in the blood such that the overall amount of the drugs in a single dosage is reduced compared to the immediate release formulation.

In some embodiments, the patient has a condition associated with excess weight including obesity, compulsive eating, and/or bulimia. In some embodiments, a patient may be selected that is not yet obese, but is overweight. Excess weight of at least 10 pounds or 10-20 pounds is associated with adverse health effects. Overweight and obesity classifications include those determined by body mass index (BMI) (calculated as weight in kilograms divided by the square of height in meters). For example, normal weight: BMI=18.5-24.9; overweight: BMI=25.0-29.9; obesity-class 1: BMI=30.0-34.9; obesity-class 2: BMI=35.0-39.9; obesity-class 3: BMI≥40.0). Of course these ranges may vary given the height, gender, and age of the subject. In other embodiments, the patient at least has a body mass index (BMI) of at least 25 or greater. In other embodiments, the patient has a BMI of at least 27 to about 30 and also has other health conditions such as hypertension, diabetes, cardiovascular disease, liver disease, and/or renal disease. In other embodiments, the patient is overweight at least 10 pounds and/or has a condition such as type II diabetes, asthma, arthritis, hypertension, high cholesterol, and/or cardiovascular disease.

Another aspect of the present application relates to a pharmaceutical composition, comprising (1) acetaminophen; (2) one or more NSAIDs; (3) one or more pharmaceutically acceptable carriers, and optionally (4) one or more active agents selected from the group consisting of anti-obesity drugs, appetite suppressants, diacylglycerol acyltransferase (DGAT) inhibitors, lipase inhibitors, and Type II diabetes agents and a pharmaceutically acceptable carrier.

In one embodiment, the pharmaceutical composition comprises a first component having an immediate-release subcomponent and an extended-release subcomponent, wherein the first component is formulated to release the subcomponents immediately after administration; and a second component comprising an immediate-release subcomponent and an extended-release subcomponent, wherein the second component is formulated for a delayed-release of the subcomponents. In some embodiments, at least one of the subcomponents in the first component or the second component comprises acetaminophen and one or more NSAIDs, and at least one of the subcomponents in the first component or the second component comprises one or more active agents selected from the group consisting of anti-obesity drugs, appetite suppressants, diacylglycerol acyltransferase (DGAT) inhibitors, lipase inhibitors, and Type II diabetes agents.

In some embodiments, each of the subcomponents in the first component or the second component comprises acetaminophen and one or more NSAIDs.

In some related embodiments, the immediate-release subcomponent and the extended-release subcomponent in the first component each comprises acetaminophen and one or more NSAIDs, and/or one or more active agents selected from the group consisting of anti-obesity drugs, appetite suppressants, diacylglycerol acyltransferase (DGAT) inhibitors, lipase inhibitors, Type II diabetes agents, and combinations thereof. In other embodiments, the immediate-release subcomponent and the extended-release subcomponent in the second component each comprises one or more active agents selected from the group consisting of anti-obesity drugs, appetite suppressants, diacylglycerol acyltransferase (DGAT) inhibitors, lipase inhibitors, Type II diabetes agents, and combinations thereof.

In some related embodiments, the second component is coated with an enteric coating.

In some related embodiments, the second component is formulated to release the subcomponents after a lag time of 1-4 or 2-4 hours or 4-8 hours following oral administration.

In some related embodiments, the extended-release subcomponent in the first component is formulated to release its active agent over a time interval of about 2-10 hours.

In some related embodiments, the extended-release subcomponent in the second component is formulated to release its active agent over a time interval of about 2-10 hours.

In some related embodiments, the active agent(s) in the immediate-release subcomponent and the extended-release subcomponents in the first component further comprises one or more anti-obesity drugs. In some embodiments, the active agent(s) in the immediate-release subcomponent and the extended-release subcomponent in the second component further comprises one or more anti-obesity drugs. In some embodiments, the active agent(s) in the immediate-release subcomponent and the extended-release subcomponent in both the first and second components each comprises one or more anti-obesity drugs.

In some related embodiments, the active agent(s) in the immediate-release subcomponent and the extended-release subcomponent in the first component further comprises one or more appetite suppressants. In some embodiments, the active agent(s) in the immediate-release subcomponent and the extended-release subcomponent in the second component further comprises one or more appetite suppressants. In some embodiments, the active agent(s) in the immediate-release subcomponent and the extended-release subcomponent in both the first and the second component each comprises one or more appetite suppressants.

In some related embodiments, the active agent(s) in the immediate-release subcomponent and the extended-release subcomponent in the first component further comprises one or more DGAT inhibitors. In some embodiments, the active agent(s) in the immediate-release subcomponent and the extended-release subcomponent in the second component further comprises one or more DGAT inhibitors. In some embodiments, the active agent(s) in the immediate-release subcomponent and the extended-release subcomponent in both the first and the second component each comprises one or more DGAT inhibitors.

In some related embodiments, the active agent(s) in the immediate-release subcomponent and the extended-release subcomponent in the first component further comprises one or more lipase inhibitors. In some embodiments, the active agent(s) in the immediate-release subcomponent and the extended-release subcomponent in the second component further comprises one or more lipase inhibitors. In some embodiments, the active agent(s) in the immediate-release subcomponent and the extended-release subcomponent in both the first and the second component each comprises one or more lipase inhibitors.

In some related embodiments, the active agent(s) in the immediate-release subcomponent and the extended-release subcomponent in the first component further comprises one or more Type II diabetes agents. In some embodiments, the active agent(s) in the immediate-release subcomponent and the extended-release subcomponent in the second component further comprises one or more Type II diabetes agents. In some embodiments, the active agent(s) in the immediate-release subcomponent and the extended-release subcomponent in both the first and the second component each comprises one or more Type II diabetes agents.

In some related embodiments, each of the immediate-release subcomponent and the extended-release subcomponent in the first component comprises acetaminophen and/or one or more NSAIDs, each in an amount between 5-2000 mg. In some embodiments, each of the immediate-release subcomponent and the extended-release subcomponent in the second component comprises acetaminophen and/or one or more NSAIDs, each in an amount between 5-2000 mg. In some embodiments, the active agent(s) in each of the immediate-release subcomponent and the extended-release subcomponent in both the first and the second component each comprises acetaminophen and/or one or more NSAIDs, each in an amount between 5-2000 mg.

In some related embodiments, the active agent(s) in the immediate-release subcomponent of the first component and the active agent(s) in the immediate-release subcomponent of the second component both comprise acetaminophen and/or one or more NSAIDs.

Another aspect of the present application relates to a pharmaceutical composition that comprises a first component comprising an immediate-release subcomponent, wherein the immediate-release subcomponent comprises one or more active agents selected from the group consisting of anti-obesity drugs, appetite suppressants, diacylglycerol acyltransferase (DGAT) inhibitors, lipase inhibitors, and Type II diabetes agents, and combinations thereof, wherein the first component is formulated to release its subcomponent immediately after oral administration; and a second component comprises an immediate-release subcomponent and an extended-release subcomponent, wherein the second component is formulated to release its subcomponent after gastric emptying, wherein the subcomponents in the second component each comprises one or more active agents selected from the group consisting of anti-obesity drugs, appetite suppressants, diacylglycerol acyltransferase (DGAT) inhibitors, lipase inhibitors, and Type II diabetes agents, and combinations thereof.

In some related embodiments, the second component is formulated to release the subcomponents after a lag time of 2-12 hours, 2-4 hours, 2-6 hours, 2-8 hours, or 4-8 hours following oral administration.

In some related embodiments, the active agents in the immediate-release subcomponent and the extended-release subcomponent of the second component comprises acetaminophen, one or more NSAIDs, and optionally one or more active agent(s) selected from the group consisting of an anti-obesity drugs, appetite suppressants, DGAT inhibitors, lipase inhibitors, Type II diabetes agents, and combinations thereof.

In some related embodiments, the first component further comprises an extended-release subcomponent, wherein the extended-release subcomponent comprises one or more active agents selected from the group consisting of anti-obesity drugs, appetite suppressants, DGAT inhibitors, lipase inhibitors, and Type II diabetes agents, and combinations thereof. In some embodiments, the Type II diabetes agents are selected from the group consisting of biguanides, DPP-IV inhibitors, glucagon-like peptide-1 (GLP-1) receptor agonists, α-glucosidase inhibitors, PPAR agonists, SGLT-2 inhibitors, and combinations thereof.

In some embodiments, the immediate-release subcomponent and the extended-release subcomponent in the second component each comprises one or more active agents selected from the group consisting of anti-obesity drugs, appetite suppressants, diacylglycerol acyltransferase (DGAT) inhibitors, lipase inhibitors, and Type II diabetes agents, and combinations thereof.

In some related embodiments, at least one of the active agents in the immediate-release subcomponent and/or the extended-release subcomponent of the first and the second components further comprises one or more active agents selected from the group consisting of anti-obesity drugs, appetite suppressants, diacylglycerol acyltransferase (DGAT) inhibitors, lipase inhibitors, and Type II diabetes agents, and combinations thereof.

In some related embodiments, the active agent in the immediate-release subcomponent and/or the extended-release subcomponent of the first component further comprises one or more active agents selected from the group consisting of anti-obesity drugs, appetite suppressants, diacylglycerol acyltransferase (DGAT) inhibitors, lipase inhibitors, and Type II diabetes agents, and combinations thereof.

In some related embodiments, the active agent in the immediate-release subcomponent and/or the extended-release subcomponent of the second component further comprises one or more active agents selected from the group consisting of anti-obesity drugs, appetite suppressants, diacylglycerol acyltransferase (DGAT) inhibitors, lipase inhibitors, and Type II diabetes agents, and combinations thereof.

Another aspect of the present application relates to a pharmaceutical composition that comprises a first component comprising an immediate-release subcomponent and an extended-release subcomponent, wherein the first component is formulated to release the subcomponents immediately after administration; and a second component comprising an immediate-release subcomponent and an extended-release subcomponent, wherein the second component is formulated for a delayed-release of the subcomponents, wherein the immediate-release subcomponent and the extended-release subcomponent in the first component each comprises an active agent comprising one or more active agents selected from the group consisting of anti-obesity drugs, appetite suppressants, diacylglycerol acyltransferase (DGAT) inhibitors, lipase inhibitors, and Type II diabetes agents, and combinations thereof, and wherein the immediate-release subcomponent and the extended-release subcomponent in the second component each comprises one or more active agents selected from the group consisting of anti-obesity drugs, appetite suppressants, diacylglycerol acyltransferase (DGAT) inhibitors, lipase inhibitors, and Type II diabetes agents, and combinations thereof, wherein the pharmaceutical composition reduces body weight in patients in need thereof.

Another aspect of the present application relates to a pharmaceutical composition that comprises an immediate-release component, an extended-release component, or both, in which each component consists of acetaminophen, ibuprofen, and optionally one or more active agents selected from the group consisting of anti-obesity drugs, appetite suppressants, diacylglycerol acyltransferase (DGAT) inhibitors, lipase inhibitors, and Type II diabetes agents, and combinations thereof.

In some embodiments, the extended-release component is formulated for extended release over a period of 0.5-24, 2-6, 6-10, 10-14, or 14-24 hours. In some embodiments, the extended-release component is formulated for extended release over a period of about 8 hours. In some embodiments, the extended-release component is coated with a delayed-release coating. In some embodiments, the delayed-release coating delays the release of the extended-release component for a period of 0.1-12, 0.5-12, 1-12, 2-12, 1-4, 2-4, 4-8 or 8-12 hours. In some embodiments, the delayed-release coating is an enteric coating. In some embodiments, the pharmaceutical composition with an immediate-release component and an extended-release component is formulated into an orally disintegrating tablet.

As used herein, the term “orally disintegrating tablet” or “orally disintegrating formulation” refers to drug tablet or formulation that rapidly disintegrates or dissolves in the oral cavity. Orally disintegrating formulations differ from traditional tablets in that they are designed to be dissolved on the tongue rather than swallowed whole. In some embodiments, the orally disintegrating formulations are designed to completely disintegrate or dissolve in the oral cavity without the aid of additional water (i.e., in saliva only) in 5, 10, 20, 30, 60, 90, 120, 180, 240, 300 or 600 seconds.

In some embodiments, the pharmaceutical composition with an immediate-release component and an extended-release component is formulated into a liquid form for oral administration. Examples of the liquid form formulation include, but are not limited to, gels, emulsions and particle suspensions. For example, the extended-release component may be formulated into a gel form that solidifies in the stomach. In some embodiments, the pharmaceutical composition with an immediate-release component and an extended-release component is formulated into a pixie pack of powder that can quickly melt on the tongue. In some embodiments, the immediate-release component or the extended release component or both further comprise one or more additional active agents selected from the group consisting of anti-obesity drugs, appetite suppressants, diacylglycerol acyltransferase (DGAT) inhibitors, lipase inhibitors, and Type II diabetes agents, and combinations thereof.

Methods of Manufacture

Another aspect of the present application relates to methods of manufacturing pharmaceutical compositions for reducing body weight.

In one embodiment, the method for manufacturing a pharmaceutical composition for reducing body weight comprises the steps of forming a first mixture comprising one or more active agents formulated for immediate release and one or more active agents formulated for extended release; coating the first mixture with a delayed release coating to form a core structure; and then coating the core structure with a second mixture comprising one or more active agents formulated for immediate release and one or more agents formulated for extended release, wherein the first mixture comprises acetaminophen and an NSAID in which each of the acetaminophen and the NSAID is formulated for both immediate release and extended release; and wherein the second mixture comprises acetaminophen and an NSAID in which each of the acetaminophen and the NSAID is formulated for both immediate release and extended release. In a preferred embodiment, the NSAID is ibuprofen.

In another embodiment, the method for manufacturing a pharmaceutical composition comprises the steps of forming a first mixture having a first active agent formulated for immediate release and a second active agent formulated for extended release; coating the first mixture with a delayed release coating to form a core structure; and then coating the core structure with a second mixture comprising a third active agent formulated for immediate release and a fourth active agent formulated for extended release, wherein at least one of the first, second, third and fourth active agents comprises acetaminophen and one or more NSAIDs, and at least one of the first, second, third and fourth active agents comprises one or more active agents selected from the group consisting of anti-obesity drugs, appetite suppressants, diacylglycerol acyltransferase (DGAT) inhibitors, lipase inhibitors, and Type II diabetes agents, and combinations thereof.

In some embodiments, at least one of the first, second, third and fourth active agents comprises (1) acetaminophen, (2) an NSAID, and optionally one or more active agents selected from the group consisting of anti-obesity drugs, appetite suppressants, DGAT inhibitors, lipase inhibitors, and Type II diabetes agents, and combinations thereof.

In some embodiments, the delayed release coating is an enteric coating. In some embodiments, the enteric coating comprises a pH-dependent polymer. In some embodiments, the delayed release coating comprises a swelling layer covered by an outer semi-permeable polymer layer. In some embodiments, the delayed release coating is formulated to release the coated material after a lag time of 0.1-12 hours, 0.5-12 hours, 1-12 hours, 2-12 hours, 1-4 hours, 2-4 hours, 2-6 hours, 2-8 hours, 4-6 hours or 4-8 hours after oral administration.

In some embodiments, the second active agent, or the fourth active agent or both comprise an active core comprising an extended-release coating or a polymeric matrix effecting diffusion controlled release.

In some embodiments, the first mixture is prepared by mixing the first active agent in liquid or powder form with the second active agent, which is formulated for extended release. As described above, the second active agent may be formulated in an extended release formulation having an active core comprised of one or more inert particles, each in the form of a bead, pellet, pill, granular particle, microcapsule, microsphere, microgranule, nanocapsule, or nanosphere coated on its surfaces with drugs in the form of e.g., a drug-containing coating or film-forming composition using, for example, fluid bed techniques or other methodologies known to those of skill in the art. The inert particle can be of various sizes, so long as it is large enough to remain undissolved. Alternatively, the active core may be prepared by granulating and milling and/or by extrusion and spheronization of a polymer composition containing the drug substance. In some embodiments, the active core comprises an extended-release coating or a polymeric matrix effecting diffusion controlled release, as described in more detail earlier. In some embodiments, the polymeric matrix is a water soluble or water-swellable matrix. In some embodiments, the second active agent is simply mixed with the first active agent. Either agent or both agents may be in the form of bead, pellet, granular particle, pill, microcapsule, microsphere, microgranule, nanocapsule or nanosphere as a powder or as a liquid suspension. In other embodiments, the second active agent form an active core that is coated with the first active agent. In some embodiments, the second active agent in the first mixture is formulated to release the active agent over a period of 2-4 hours, 2-6 hours, 2-8 hours or 2-10 hours.

In some embodiments, the second active agent is kept in a compartment partially or completely separate from the first active agent. In other embodiments, the first mixture is formed by keeping the second active agent in a compartment partially or completely separated from the first active agent.

The first mixture is then coated with a delayed release coating to form a core structure. In some embodiments, the delayed release coating is an enteric coating. In some embodiments, the enteric coating comprises a pH-dependent polymer that maintains its structure integrity at low pH, such as the pH in the stomach (normally in the range of 1.5-3.5). In some embodiments, the term “low pH” refers to a pH value of 4.0, 3.5, 3.0, 2.5, 2.0, 1.5, 1.0 or lower. In some embodiments, the enteric coating comprises one or more pH-dependent polymers and one or more polysaccharides that are resistant to erosion in both the stomach and intestine, thus allowing the release of the first mixture only in the colon. In some embodiments, the delayed release coating comprises two or more layers of coating. In some embodiments, the delayed release coating comprises a swelling layer and an outer semi-permeable polymer layer that covers the swelling layer.

In the next step, the coated core structure is re-coated with a second mixture that comprises a third active agent formulated for immediate release and a fourth active agent formulated for extended release. In some embodiments, the second mixture is prepared by mixing the third active agent in liquid or powder form with the fourth active agent, which is formulated for extended release. The fourth active agent may be formulated in an extended release formulation having an active core comprised of one or more inert particles, each in the form of a bead, pellet, pill, granular particle, microcapsule, microsphere, microgranule, nanocapsule, or nanosphere coated on its surfaces with drugs in the form of e.g., a drug-containing coating or film-forming composition using, for example, fluid bed techniques or other methodologies known to those of skill in the art. The inert particle can be of various sizes, so long as it is large enough to remain poorly dissolved. Alternatively, the active core may be prepared by granulating and milling and/or by extrusion and spheronization of a polymer composition containing the drug substance. In some embodiments, the active core comprises an extended-release coating or a polymeric matrix effecting diffusion controlled release, as described in more detail earlier. In some embodiments, the polymeric matrix is a water soluble or water-swellable matrix. In some embodiments, the fourth active agent is simply mixed with the third active agent. Either agent or both agents may be in the form of bead, pellet, granular particle, pill, microcapsule, microsphere, microgranule, nanocapsule or nanosphere as a powder or as a liquid suspension.

In other embodiments, the coated core structure is re-coated first with the fourth active agent, and then coated with the third active agent. In some embodiments, the fourth active agent is formulated to release the active agent over a period of 2-4 hours, 2-6 hours, 2-8 hours or 2-10 hours.

In some embodiments, the fourth active agent is kept in a compartment partially or completely separated from the third active agent. In other embodiments, the second mixture is formed by keeping the fourth active agent in a compartment partially or completely separated from the third active agent.

In other embodiments, the method comprises the steps of forming a core structure comprising a first active agent formulated for immediate release and a second active agent formulated for extended release, coating the core structure with a delayed release coating to form a coated core structure, and mixing the coated core structure with a third active agent formulated for immediate release and a fourth active agent formulated for extended release. The first, second, third and fourth active agents can be any of the active agents described above. In one embodiment, the first and second active agents comprise acetaminophen and an NSAID, and the third and fourth active agents are selected from the group consisting of anti-obesity drugs, appetite suppressants, DGAT inhibitors, lipase inhibitors, and Type II diabetes agents, and combinations thereof. In some embodiments, the method further comprises the step of preparing a dosage form with the final mixture. In some embodiments, the dosage form is in a tablet form. In some embodiments, the dosage form is in an orally disintegrating form, e.g., orally disintegrating tablet form. In some embodiments, the dosage form is in a beads-in-a-capsule form. In some embodiments, the dosage form is in a liquid (e.g., emulsion) form.

In other embodiments, the method comprises the steps of forming a core structure comprising a first active agent formulated for immediate release and a second active agent formulated for extended release, coating the core structure with a delayed release coating to form a coated core structure, mixing the coated core structure with a third agent formulated for immediate release and a fourth agent formulated for extended release.

Another aspect of the present application relates to a method for manufacturing a pharmaceutical composition for reducing body weight comprising the step of forming a core structure comprising a first active agent formulated for immediate release and a second active agent formulated for extended release; coating the core structure with a delayed release coating to form a coated core structure; mixing the coated core structure with a third active agent formulated for immediate release and a fourth active agent formulated for extended release to form a final mixture, and compressing the final mixture into a tablet. In some embodiments, the first and second active agents comprise acetaminophen and an NSAID, and the third and fourth active agents are selected from the group consisting of anti-obesity drugs, appetite suppressants, DGAT inhibitors, lipase inhibitors, and Type II diabetes agents.

In some embodiments, the first, second, third, and fourth active agents comprise: (1) acetaminophen; (2) an NSAID; and (3) at least two active agents selected from the group consisting of anti-obesity drugs, appetite suppressants, DGAT inhibitors, lipase inhibitors, and Type II diabetes agents.

Another aspect of the present application relates to a method for manufacturing a pharmaceutical composition for reducing body weight comprising the steps of forming a core structure comprising a first active agent formulated for immediate release and a second active agent formulated for extended release; coating the core structure with a delayed release coating to form a coated core structure; coating the coated core structure with a third active agent formulated for immediate release to form a double-coated core structure. In some embodiments, wherein the first and second active agents comprises acetaminophen and an NSAID, respectively, and at least one of the three active agents is selected from the group consisting of anti-obesity drugs, appetite suppressants, DGAT inhibitors, lipase inhibitors, and Type II diabetes agents.

In some embodiments, the first, second and third active agents comprises: (1) acetaminophen; (2) an NSAID, such as ibuprofen; and (3) an active agent selected from the group consisting of anti-obesity drugs, appetite suppressants, DGAT inhibitors, lipase inhibitors, and Type II diabetes agents.

Another aspect of the present application relates to a method for manufacturing a pharmaceutical composition for reducing body weight comprising the steps of forming a core structure comprising acetaminophen and one or more NSAIDs formulated for extended-release, and coating the core structure with a coating layer comprising acetaminophen and one or more NSAIDs, wherein the coating layer is formulated for immediate release and wherein either the core structure, the coating layer, or both, further comprise an anti-obesity drug, appetite suppressant, DGAT inhibitor, lipase inhibitor, a Type II diabetes agent, or a combination thereof.

In some embodiments, the core structure is first coated with a delayed-release coating and then coated with a coating layer comprising acetaminophen and one or more NSAIDs, which is formulated for immediate release.

In some embodiments, the method comprises the steps of forming a first mixture comprising acetaminophen and one or more NSAIDs formulated for extended-release, forming a second mixture comprising acetaminophen and one or more NSAIDs formulated for immediate-release, and combining the first mixture and the second mixture to form a final mixture, wherein either the first mixture or the second mixture or both further comprise an anti-obesity drug, appetite suppressant, DGAT inhibitor, lipase inhibitor, a Type II diabetes agent, or a combination thereof.

In some embodiments, the first mixture, the second mixture and the final mixture are mixtures of solid materials. In some embodiments, the final mixture is in powder or granulate form. In some embodiments, the method further comprises the step of pressing the final mixture into a tablet form. In some embodiments, the final mixture is in a liquid, gel or emulsion form.

For example, the extended-release component may be formulated into a gel form that solidifies in the stomach. In some embodiment, the pharmaceutical composition with an immediate-release component and an extended-release component is formulated into a pixie pack of powder that can quickly melt on the tongue. In some embodiments, the pharmaceutical composition with an immediate-release component and an extended-release component is formulated into an orally disintegrating tablet using loose compression tableting. In loose compression, orally disintegrating formulation is compressed at much lower forces (4-20 kN) than traditional tablets. In some embodiments, the orally disintegrating formulation contains some form of sugar, such as mannitol, to improve mouth feel. In some embodiments, the orally disintegrating tablet is produced using lyophilized orally disintegrating formulation.

The present invention is further illustrated by the following example which should not be construed as limiting. The contents of all references, patents, and published patent applications cited throughout this application are incorporated herein by reference.

Example 1-Weight loss following administration of acetaminophen in combination with an NSAID.

The results of a recently concluded Phase 2 placebo-controlled FDA approved clinical trial included data on 39 patients over a two-week period. 9 of the patients took placebo pills about 30 minutes before bedtime. 30 of the patients took 1, 2, or 3 pills each containing a combined immediate and extended release formulation of acetaminophen (325 mg) and ibuprofen (150 mg). Each patient took 3 pills, so in about half the cases it was a mixture of placebo and the drug. Over the 2 weeks those on placebo gained an average of 0.1 pounds (extrapolates to 2.6 pounds/year) while those using either of the 3 doses of the drug lost an average of 0.5 pounds (extrapolates to 13 pounds per year). The women in the trial weighed an average of 184 pounds and the men weighed an average of 196 pounds. The mechanism of action is believed to be improved sleep, which leads to less fatigue and depression. Since fatigue and depression lead to overeating and lack of exercise, presumably reducing fatigue and depression will reduce overeating and encourage more exercise.

1. A method for reducing body weight in a subject, comprising:
administering to a subject in need thereof a pharmaceutical composition comprising acetaminophen in an amount sufficient for reducing body weight,
wherein the acetaminophen is administered orally at a daily dose of 5 mg to 2000 mg, and
wherein the composition is formulated in a controlled release formulation.

2. The method of claim 1, wherein the acetaminophen is administered orally at a daily dose of 50 mg to 500 mg.

3. The method of claim 1, wherein the acetaminophen is administered orally at a daily dose of 250 mg to 500 mg.

4. The method of claim 1, wherein the acetaminophen is formulated in an extended-release formulation.

5. The method of claim 1, wherein the extended release formulation comprises acetaminophen embedded in a matrix of insoluble substance(s).

6. The method of claim 1, wherein the extended-release formulation comprises a water soluble or water-swellable matrix-forming polymer.

7. The method of claim 1, wherein the extended-release formulation comprises a polymer controlling release of acetaminophen by dissolution-controlled release.

8. The method of claim 1, wherein the extended-release formulation is coated with an enteric coating.

9. The method of claim 1, wherein the composition further comprises one or more additional active agents selected from the group consisting of anti-obesity drugs, appetite suppressants, diacylglycerol acyltransferase (DGAT) inhibitors, lipase inhibitors, nonsteroidal anti-inflammatory drugs (NSAID), Type II diabetes agents, anti-insomnia agents and combinations thereof.

10. The method of claim 1, wherein the composition is formulated to release the one or more additional active agents by immediate release, extended release, delayed release, or a combination thereof.

11. The method of claim 9, wherein the one or more additional active agents comprise at least one anti-obesity drug.

12. The method of claim 9, wherein the one or more additional active agents comprise at least one appetite suppressant.

13. The method of claim 9, wherein the one or more additional active agents comprise at least one DGAT inhibitor.

14. The method of claim 9, wherein the one or more additional active agents comprise at least one lipase inhibitor.

15. The method of claim 9, wherein the one or more additional active agents comprise at least one NSAID.

16. The method of claim 9, wherein the one or more additional active agents comprise at least one Type II diabetes agent.

17. The method of claim 16, wherein the NSAID is ibuprofen.

18. The method of claim 18, wherein each of the acetaminophen and ibuprofen is administered in an extended-release formulation.

19. The method of claim 18, wherein the acetaminophen and ibuprofen are the only active therapeutic agents in the composition.

20. A method for manufacturing a pharmaceutical composition for reducing body weight, comprising the steps of:
forming a first mixture comprising one or more active agents formulated for immediate release and one or more active agents formulated for extended release;
coating the first mixture with a delayed release coating to form a core structure; and then
coating the core structure with a second mixture comprising one or more active agents formulated for immediate release and one or more agents formulated for extended release,
wherein the first mixture comprises acetaminophen and an NSAID in which each of the acetaminophen and the NSAD is formulated for both immediate release and extended release; and
wherein the second mixture comprises acetaminophen and an NSAID in which each of the acetaminophen and the NSAID is formulated for both immediate release and extended release.
